SEQUENCE LISTING (55 sequences)

SEQ ID NO: 1 - protein, 30 aa, Artificial Sequence
  Description of Artificial Sequence Synthetic polypeptide
PAICQRATATLGTVGSNTSGTTEIEACILL

SEQ ID NO: 2 - protein, 15 aa, Artificial Sequence
  Description of Artificial Sequence Synthetic peptide
IALILEPICCQERAA

SEQ ID NO: 3 - protein, 15 aa, Artificial Sequence
  Description of Artificial Sequence Synthetic peptide
IALILEPICCQERAA

SEQ ID NO: 4 - protein, 15 aa, Artificial Sequence
  Description of Artificial Sequence Synthetic peptide
  MOD_RES (8)..(8): Sarcosine
IALILEPXCCQERAA

SEQ ID NO: 5 - protein, 15 aa, Artificial Sequence
  Description of Artificial Sequence Synthetic peptide
  MOD_RES (9)..(9): Sarcosine
IALILEPIXCQERAA

SEQ ID NO: 6 - protein, 15 aa, Artificial Sequence
  Description of Artificial Sequence Synthetic peptide
  MOD_RES (6)..(6): (R)-2-(7′-octenyl)alanine
  SITE (6)..(13): May or may not be stapled
  MOD_RES (8)..(8): Sarcosine
  MOD_RES (13)..(13): (S)-2-(4′-pentenyl)alanine
IALILAPXCCQEAAA

SEQ ID NO: 7 - protein, 15 aa, Artificial Sequence
  Description of Artificial Sequence Synthetic peptide
  MOD_RES (2)..(2): (R)-2-(7′-octenyl)alanine
  SITE (2)..(9): May or may not be stapled
  MOD_RES (8)..(8): Sarcosine
  MOD_RES (9)..(9): (S)-2-(4′-pentenyl)alanine
IALILEPXACQERAA

SEQ ID NO: 8 - protein, 15 aa, Artificial Sequence
  Description of Artificial Sequence Synthetic peptide
  MOD_RES (8)..(8): Sarcosine
  MOD_RES (11)..(11): (S)-2-(4′-pentenyl)alanine
  SITE (11)..(15): May or may not be stapled
  MOD_RES (15)..(15): (S)-2-(4′-pentenyl)alanine
IALILEPXCCAERAA

SEQ ID NO: 9 - protein, 15 aa, Artificial Sequence
  Description of Artificial Sequence Synthetic peptide
  MOD_RES (8)..(8): Sarcosine
  MOD_RES (10)..(10): (S)-2-(4′-pentenyl)alanine
  SITE (10)..(14): May or may not be stapled
  MOD_RES (14)..(14): (S)-2-(4′-pentenyl)alanine
IALILEPXCAQERAA

SEQ ID NO: 10 - protein, 15 aa, Artificial Sequence
  Description of Artificial Sequence Synthetic peptide
  MOD_RES (8)..(8): Sarcosine
  MOD_RES (9)..(9): (S)-2-(4′-pentenyl)alanine
  SITE (9)..(13): May or may not be stapled
  MOD_RES (13)..(13): (S)-2-(4′-pentenyl)alanine
IALILEPXACQEAAA

SEQ ID NO: 11 - protein, 15 aa, Artificial Sequence
  Description of Artificial Sequence Synthetic peptide
  MOD_RES (6)..(6): (S)-2-(4′-pentenyl)alanine
  SITE (6)..(10): May or may not be stapled
  MOD_RES (8)..(8): Sarcosine
  MOD_RES (10)..(10): (S)-2-(4′-pentenyl)alanine
IALILAPXCAQERAA

SEQ ID NO: 12 - protein, 15 aa, Artificial Sequence
  Description of Artificial Sequence Synthetic peptide
  MOD_RES (1)..(1): D-amino acid
  MOD_RES (8)..(8): Sarcosine
IALILEPXCCQERAA

SEQ ID NO: 13 - protein, 15 aa, Artificial Sequence
  Description of Artificial Sequence Synthetic peptide
  MOD_RES (2)..(2): D-amino acid
  MOD_RES (8)..(8): Sarcosine
IALILEPXCCQERAA

SEQ ID NO: 14 - protein, 15 aa, Artificial Sequence
  Description of Artificial Sequence Synthetic peptide
  MOD_RES (4)..(4): D-amino acid
  MOD_RES (8)..(8): Sarcosine
IALILEPXCCQERAA

SEQ ID NO: 15 - protein, 15 aa, Artificial Sequence
  Description of Artificial Sequence Synthetic peptide
  MOD_RES (5)..(5): D-amino acid
  MOD_RES (8)..(8): Sarcosine
IALILEPXCCQERAA

SEQ ID NO: 16 - protein, 15 aa, Artificial Sequence
  Description of Artificial Sequence Synthetic peptide
  MOD_RES (6)..(6): D-amino acid
  MOD_RES (8)..(8): Sarcosine
IALILEPXCCQERAA

SEQ ID NO: 17 - protein, 15 aa, Artificial Sequence
  Description of Artificial Sequence Synthetic peptide
  MOD_RES (7)..(7): D-amino acid
  MOD_RES (8)..(8): Sarcosine
IALILEPXCCQERAA

SEQ ID NO: 18 - protein, 15 aa, Artificial Sequence
  Description of Artificial Sequence Synthetic peptide
  MOD_RES (8)..(8): Sarcosine
  MOD_RES (9)..(9): D-amino acid
IALILEPXCCQERAA

SEQ ID NO: 19 - protein, 15 aa, Artificial Sequence
  Description of Artificial Sequence Synthetic peptide
  MOD_RES (8)..(8): Sarcosine
  MOD_RES (10)..(10): D-amino acid
IALILEPXCCQERAA

SEQ ID NO: 20 - protein, 15 aa, Artificial Sequence
  Description of Artificial Sequence Synthetic peptide
  MOD_RES (8)..(8): Sarcosine
  MOD_RES (11)..(11): D-amino acid
IALILEPXCCQERAA

SEQ ID NO: 21 - protein, 15 aa, Artificial Sequence
  Description of Artificial Sequence Synthetic peptide
  MOD_RES (8)..(8): Sarcosine
  MOD_RES (12)..(12): D-amino acid
IALILEPXCCQERAA

SEQ ID NO: 22 - protein, 15 aa, Artificial Sequence
  Description of Artificial Sequence Synthetic peptide
  MOD_RES (8)..(8): Sarcosine
  MOD_RES (13)..(13): D-amino acid
IALILEPXCCQERAA

SEQ ID NO: 23 - protein, 15 aa, Artificial Sequence
  Description of Artificial Sequence Synthetic peptide
  MOD_RES (8)..(8): Sarcosine
  MOD_RES (14)..(14): D-amino acid
IALILEPXCCQERAA

SEQ ID NO: 24 - protein, 15 aa, Artificial Sequence
  Description of Artificial Sequence Synthetic peptide
  MOD_RES (8)..(8): Sarcosine
  MOD_RES (15)..(15): D-amino acid
IALILEPXCCQERAA

SEQ ID NO: 25 - protein, 15 aa, Artificial Sequence
  Description of Artificial Sequence Synthetic peptide
  MOD_RES (4)..(4): (R)-2-(7′-octenyl)alanine
  SITE (4)..(11): May or may not be stapled
  MOD_RES (8)..(8): Sarcosine
  MOD_RES (11)..(11): (S)-2-(4′-pentenyl)alanine
IALALEPXCCAERAA

SEQ ID NO: 26 - protein, 15 aa, Artificial Sequence
  Description of Artificial Sequence Synthetic peptide
  MOD_RES (5)..(5): (R)-2-(7′-octenyl)alanine
  SITE (5)..(12): May or may not be stapled
  MOD_RES (8)..(8): Sarcosine
  MOD_RES (12)..(12): (S)-2-(4′-pentenyl)alanine
IALIAEPXCCQARAA

SEQ ID NO: 27 - protein, 15 aa, Artificial Sequence
  Description of Artificial Sequence Synthetic peptide
  MOD_RES (7)..(7): (R)-2-(7′-octenyl)alanine
  SITE (7)..(15): May or may not be stapled
  MOD_RES (8)..(8): Sarcosine
  MOD_RES (15)..(15): (S)-2-(4′-pentenyl)alanine
IALILEAXCCQERAA

SEQ ID NO: 28 - protein, 15 aa, Artificial Sequence
  Description of Artificial Sequence Synthetic peptide
  MOD_RES (1)..(1): (S)-2-(4′-pentenyl)alanine
  SITE (1)..(5): May or may not be stapled
  MOD_RES (5)..(5): (S)-2-(4′-pentenyl)alanine
  MOD_RES (8)..(8): Sarcosine
AALIAEPXCCQERAA

SEQ ID NO: 29 - protein, 15 aa, Artificial Sequence
  Description of Artificial Sequence Synthetic peptide
  MOD_RES (3)..(3): (S)-2-(4′-pentenyl)alanine
  SITE (3)..(7): May or may not be stapled
  MOD_RES (7)..(7): (S)-2-(4′-pentenyl)alanine
  MOD_RES (8)..(8): Sarcosine
IAAILEAXCCQERAA

SEQ ID NO: 30 - protein, 15 aa, Artificial Sequence
  Description of Artificial Sequence Synthetic peptide
  MOD_RES (5)..(5): (S)-2-(4′-pentenyl)alanine
  SITE (5)..(9): May or may not be stapled
  MOD_RES (8)..(8): Sarcosine
  MOD_RES (9)..(9): (S)-2-(4′-pentenyl)alanine
IALIAEPXACQERAA

SEQ ID NO: 31 - protein, 15 aa, Artificial Sequence
  Description of Artificial Sequence Synthetic peptide
  MOD_RES (7)..(7): (S)-2-(4′-pentenyl)alanine
  SITE (7)..(11): May or may not be stapled
  MOD_RES (8)..(8): Sarcosine
  MOD_RES (11)..(11): (S)-2-(4′-pentenyl)alanine
IALILEAXCCAERAA

SEQ ID NO: 32 - protein, 15 aa, Artificial Sequence
  Description of Artificial Sequence Synthetic peptide
  MOD_RES (8)..(8): Sarcosine
  MOD_RES (9)..(9): D-amino acid
  MOD_RES (10)..(10): (S)-2-(4′-pentenyl)alanine
  SITE (10)..(14): May or may not be stapled
  MOD_RES (14)..(14): (S)-2-(4′-pentenyl)alanine
IALILEPXCAQERAA

SEQ ID NO: 33 - protein, 15 aa, Artificial Sequence
  Description of Artificial Sequence Synthetic peptide
  MOD_RES (8)..(8): Sarcosine
  MOD_RES (10)..(10): (S)-2-(4′-pentenyl)alanine
  SITE (10)..(14): May or may not be stapled
  MOD_RES (12)..(12): D-amino acid
  MOD_RES (14)..(14): (S)-2-(4′-pentenyl)alanine
IALILEPXCAQERAA

SEQ ID NO: 34 - protein, 15 aa, Artificial Sequence
  Description of Artificial Sequence Synthetic peptide
  MOD_RES (8)..(8): Sarcosine
  MOD_RES (10)..(10): (S)-2-(4′-pentenyl)alanine
  SITE (10)..(14): May or may not be stapled
  MOD_RES (14)..(14): (S)-2-(4′-pentenyl)alanine
  MOD_RES (15)..(15): D-amino acid
IALILEPXCAQERAA

SEQ ID NO: 35 - protein, 15 aa, Artificial Sequence
  Description of Artificial Sequence Synthetic peptide
  MOD_RES (1)..(1): (R)-2-(7′-octenyl)alanine
  SITE (1)..(8): May or may not be stapled
  MOD_RES (8)..(8): (S)-2-(4′-pentenyl)alanine
  MOD_RES (9)..(9): Sarcosine
AALILEPAXCQERAA

SEQ ID NO: 36 - protein, 15 aa, Artificial Sequence
  Description of Artificial Sequence Synthetic peptide
  MOD_RES (4)..(4): (R)-2-(7′-octenyl)alanine
  SITE (4)..(11): May or may not be stapled
  MOD_RES (9)..(9): Sarcosine
  MOD_RES (11)..(11): (S)-2-(4′-pentenyl)alanine
IALALEPIXCAERAA

SEQ ID NO: 37 - protein, 15 aa, Artificial Sequence
  Description of Artificial Sequence Synthetic peptide
  MOD_RES (5)..(5): (R)-2-(7′-octenyl)alanine
  SITE (5)..(12): May or may not be stapled
  MOD_RES (9)..(9): Sarcosine
  MOD_RES (12)..(12): (S)-2-(4′-pentenyl)alanine
IALIAEPIXCQARAA

SEQ ID NO: 38 - protein, 15 aa, Artificial Sequence
  Description of Artificial Sequence Synthetic peptide
  MOD_RES (6)..(6): (R)-2-(7′-octenyl)alanine
  SITE (6)..(13): May or may not be stapled
  MOD_RES (9)..(9): Sarcosine
  MOD_RES (13)..(13): (S)-2-(4′-pentenyl)alanine
IALILAPIXCQEAAA

SEQ ID NO: 39 - protein, 15 aa, Artificial Sequence
  Description of Artificial Sequence Synthetic peptide
  MOD_RES (1)..(1): (S)-2-(4′-pentenyl)alanine
  SITE (1)..(5): May or may not be stapled
  MOD_RES (5)..(5): (S)-2-(4′-pentenyl)alanine
  MOD_RES (9)..(9): Sarcosine
AALIAEPIXCQERAA

SEQ ID NO: 40 - protein, 15 aa, Artificial Sequence
  Description of Artificial Sequence Synthetic peptide
  MOD_RES (3)..(3): (S)-2-(4′-pentenyl)alanine
  SITE (3)..(7): May or may not be stapled
  MOD_RES (7)..(7): (S)-2-(4′-pentenyl)alanine
  MOD_RES (9)..(9): Sarcosine
IAAILEAIXCQERAA

SEQ ID NO: 41 - protein, 15 aa, Artificial Sequence
  Description of Artificial Sequence Synthetic peptide
  MOD_RES (9)..(9): Sarcosine
  MOD_RES (11)..(11): (S)-2-(4′-pentenyl)alanine
  SITE (11)..(15): May or may not be stapled
  MOD_RES (15)..(15): (S)-2-(4′-pentenyl)alanine
IALILEPIXCAERAA

SEQ ID NO: 42 - protein, 15 aa, Artificial Sequence
  Description of Artificial Sequence Synthetic peptide
  MOD_RES (1)..(1): D-amino acid
  MOD_RES (9)..(9): Sarcosine
IALILEPIXCQERAA

SEQ ID NO: 43 - protein, 15 aa, Artificial Sequence
  Description of Artificial Sequence Synthetic peptide
  MOD_RES (2)..(2): D-amino acid
  MOD_RES (9)..(9): Sarcosine
IALILEPIXCQERAA

SEQ ID NO: 44 - protein, 15 aa, Artificial Sequence
  Description of Artificial Sequence Synthetic peptide
  MOD_RES (3)..(3): D-amino acid
  MOD_RES (9)..(9): Sarcosine
IALILEPIXCQERAA

SEQ ID NO: 45 - protein, 15 aa, Artificial Sequence
  Description of Artificial Sequence Synthetic peptide
  MOD_RES (4)..(4): D-amino acid
  MOD_RES (9)..(9): Sarcosine
IALILEPIXCQERAA

SEQ ID NO: 46 - protein, 15 aa, Artificial Sequence
  Description of Artificial Sequence Synthetic peptide
  MOD_RES (5)..(5): D-amino acid
  MOD_RES (9)..(9): Sarcosine
IALILEPIXCQERAA

SEQ ID NO: 47 - protein, 15 aa, Artificial Sequence
  Description of Artificial Sequence Synthetic peptide
  MOD_RES (6)..(6): D-amino acid
  MOD_RES (9)..(9): Sarcosine
IALILEPIXCQERAA

SEQ ID NO: 48 - protein, 15 aa, Artificial Sequence
  Description of Artificial Sequence Synthetic peptide
  MOD_RES (7)..(7): D-amino acid
  MOD_RES (9)..(9): Sarcosine
IALILEPIXCQERAA

SEQ ID NO: 49 - protein, 15 aa, Artificial Sequence
  Description of Artificial Sequence Synthetic peptide
  MOD_RES (8)..(8): D-amino acid
  MOD_RES (9)..(9): Sarcosine
IALILEPIXCQERAA

SEQ ID NO: 50 - protein, 15 aa, Artificial Sequence
  Description of Artificial Sequence Synthetic peptide
  MOD_RES (9)..(9): Sarcosine
  MOD_RES (11)..(11): D-amino acid
IALILEPIXCQERAA

SEQ ID NO: 51 - protein, 15 aa, Artificial Sequence
  Description of Artificial Sequence Synthetic peptide
  MOD_RES (9)..(9): Sarcosine
  MOD_RES (12)..(12): D-amino acid
IALILEPIXCQERAA

SEQ ID NO: 52 - protein, 15 aa, Artificial Sequence
  Description of Artificial Sequence Synthetic peptide
  MOD_RES (9)..(9): Sarcosine
  MOD_RES (13)..(13): D-amino acid
IALILEPIXCQERAA

SEQ ID NO: 53 - protein, 15 aa, Artificial Sequence
  Description of Artificial Sequence Synthetic peptide
  MOD_RES (9)..(9): Sarcosine
  MOD_RES (15)..(15): D-amino acid
IALILEPIXCQERAA

SEQ ID NO: 54 - protein, 15 aa, Artificial Sequence
  Description of Artificial Sequence Synthetic peptide
  MOD_RES (8)..(8): Sarcosine
  MOD_RES (9)..(9): D-amino acid
IALILEPXCCQERRA

SEQ ID NO: 55 - protein, 14 aa, Artificial Sequence
  Description of Artificial Sequence Synthetic peptide
  MOD_RES (8)..(8): Sarcosine
IALILEPXCCQERA

(19) United States
(12) Patent Application Publication
(10) Pub. No.: US 2023/0272012 A1
(43) Pub. Date: Aug. 31, 2023

(54) PEPTIDES AND METHODS OF USE

(71) Applicant: REALTA LIFE SCIENCES, INC., Norfolk, VA (US)

(72) Inventors: Neel K. KRISHNA, Norfolk, VA (US); Kenji CUNNION, Norfolk, VA (US)

(73) Assignee: REALTA LIFE SCIENCES, INC., Norfolk, VA (US)

(21) Appl. No.: 18/034,731

(22) PCT Filed: Nov. 1, 2021
(86) PCT No.: PCT/US2021/057504

§ 371 Date: May 1, 2023

Related U.S. Application Data
(60) Provisional application No. 63/108,732, filed on Nov. 2, 2020.

(51) Int. Cl. C07K 14/00; C07K 7/08; A61P 9/00; A61P 37/04

(52) U.S. Cl. CPC C07K 14/001; C07K 7/08; A61P 9/00; A61P 37/04

(57) ABSTRACT

The present invention provides synthetic peptides. The invention is directed to modifications of a synthetic peptide of 15 amino acids from the Polar Assortant (PA) peptide, which is a scrambled peptide derived from human astrovirus protein. In some embodiments, the invention is directed to peptides that are modifications of PA including sarcosine substitutions at certain amino acid positions that are stapled and/or have D-enantiomeric substitutions of certain amino acids. The invention further provides methods of selecting at least one synthetic peptide for treating various conditions.

### CROSS-REFERENCE TO RELATED APPLICATIONS

This application claims priority to U.S. Provisional Application No. 63/108,732, filed on 2 Nov. 2020, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Oct. 28, 2021, is named 251110_000158_SL.txt and is 25,380 bytes in size.

### BACKGROUND OF THE INVENTION

### 1. Field of the Invention

Embodiments of the present invention relates generally to synthetic peptides and uses thereof for therapy and diagnostics, and more specifically to stapled forms of the synthetic peptides, alone or in combination with D-enantiomers of certain amino acids of the synthetic peptides.

### 2. Background

The Complement System

The complement system, an essential component of the innate immune system, plays a critical role as a defense mechanism against invading pathogens, primes adaptive immune responses, and helps remove immune complexes and apoptotic cells. Three different pathways comprise the complement system: the classical pathway, the lectin pathway and alternative pathway. C1q and mannose-binding lectin (MBL) are the structurally related recognition molecules of the classical and lectin pathways, respectively. Whereas IgM or clustered IgG serve as the principal ligands for C1q, MBL recognizes polysaccharides such as mannan. Ligand binding by C1q and MBL results in the sequential activation of C4 and C2 to form the classical and lectin pathway C3-convertase, respectively. In contrast, alternative pathway activation does not require a recognition molecule, but can amplify C3 activation initiated by the classical or lectin pathways. Activation of any of these three pathways results in the formation of inflammatory mediators (C3a and C5a) and the membrane attack complex (MAC), which causes cellular lysis.

While the complement system plays a critical role in many protective immune functions, complement activation is a significant mediator of tissue damage in a wide range of autoimmune and inflammatory disease processes. (Ricklin and Lambris, “Complement-targeted therapeutics.” Nat Biotechnol 2007; 25(11):1265-75).

A need exists for complement regulators. On the one hand, the complement system is a vital host defense against pathogenic organisms. On the other hand, its unchecked activation can cause devastating host cell damage. Currently, despite the known morbidity and mortality associated with complement dysregulation in many disease processes, including autoimmune diseases such as systemic lupus erythematosus, myasthenia gravis, and multiple sclerosis, only two anti-complement therapies have recently been approved for use in humans:) eculizumab (Soliris™) and 2) ultomiris (Ravulizumab™) two humanized, long-acting monoclonal antibodies against C5 used in the treatment of paroxysmal nocturnal hemoglobinuria (PNH) and atypical hemolytic uremic syndrome (aHUS). PNH and aHUS are orphan diseases in which very few people are afflicted. Currently, no complement regulators are approved for the more common disease processes in which dysregulated complement activation plays a pivotal role. Dysregulated complement activation can play a role in both chronic disease indications and acute disease indications.

Developing peptides to inhibit classical, lectin and alternative pathways of the complement system is needed, as each of these three pathways have been demonstrated to contribute to numerous autoimmune and inflammatory disease processes. Specific blockade of classical and lectin pathways is particularly needed, as both of these pathways have been implicated in ischemia reperfusion-induced injury and other diseases in many animal models. Humans with alternative pathway deficiencies suffer severe bacterial infections. Thus, a functional alternative pathway is essential for immune surveillance against invading pathogens.

Naturally occurring peptides are essential signaling molecules that play critical physiological roles in human biology in the form of neurotransmitters, hormones, growth factors and anti-microbials [1]. Given their intrinsic specificity and efficient properties, this class of molecules have received considerable attention as human therapeutics for a variety of disease indications, with over 60 approved for therapeutic use in the US, Europe and/or Japan and 155 currently in clinical development as of March, 2018 [2]. The advantageous properties of peptides provides a significant advantage over small molecules (<500 Da) which often suffer from toxicity and off-target effects. Additionally, compared to large protein-based molecules such as humanized monoclonal antibodies, peptides typically enjoy low costs of manufacturing and in many cases can be synthesized chemically, thus avoiding costly and complex production and purification. Often, naturally occurring peptides cannot be directly translated into therapeutic use due to sub-optimal chemical and physical stability and poor pharmacokinetics (half-life). Thus, a number of technological approaches to rationally design peptides into more druggable molecules suitable for human administration are frequently employed.

The inventors have identified a novel family of peptides known as PIC1 (also referred to as EPICC peptides). The PIC1 peptides possess multiple anti-inflammatory properties including inhibition of the classical pathway of complement, myeloperoxidase (MPO) inhibition, neutrophil extracellular trap (NET) inhibition as well as intrinsic antioxidant and anti-microbial activity [3-8]. The precursor to the PIC1 peptides were initially based upon the finding that the 787 amino acid capsid protein sequence of human astrovirus type 1, a non-enveloped icosahedral RNA virus that is an endemic pathogen causing gastroenteritis in human infants [9], could inhibit activation of the classical pathway of complement [10].

The PIC1/EPICC family of molecules comprise a collection of rationally designed peptides with several anti-inflammatory functional properties including inhibition of the classical pathway of complement, myeloperoxidase inhibition, neutrophil extracellular trap inhibition and antioxidant activity. The original PIC1 peptide is a 15 amino acid peptide sequence, IALILEPICCQERAA (SEQ ID NO: 2), derived from a scrambled astroviral coat protein. The original PIC1 peptide has been modified with a C-terminal monodisperse 24-mer PEGylated moiety (IALILEPICCQERAA-dPEG24; PA-dPEG24; SEQ ID NO: 3), increasing its aqueous solubility. A sarcosine substitution scan of SEQ ID NO: 3 revealed that replacement of isoleucine at position 8 or cysteine at position 9 with sarcosine resulted in two peptides, IALILEP(Sar)CCQERAA (PA-I8Sar; SEQ ID NO: 4) and IALILEPI(Sar)CQERAA (PA-C9Sar; SEQ ID NO: 5), were water soluble without PEGylation (as described in U.S. Pat. No. 10,005,818). Additional variants based on the PA-I8Sar and PA-I9Sar molecules were constructed, including stapled forms of the peptides and/or one or more D-enantiomeric substitutions of certain amino acid positions.

### BRIEF SUMMARY OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to synthetic peptides that are stapled and/or include one or more D-enantiomeric forms of the amino acids.

In one aspect, the present invention provides synthetic peptides that regulate the complement system and methods of using these peptides. Specifically, in some embodiments, the synthetic peptides can bind, regulate and inactivate C1 and MBL, and therefore can efficiently inhibit classical and lectin pathway activation at its earliest point while leaving the alternative pathway intact. These peptides are of therapeutic value for selectively regulating and inhibiting C1 and MBL activation without affecting the alternative pathway and can be used for treating diseases mediated by dysregulated activation of the classical and lectin pathways. In other embodiments, the peptides regulate classical pathway activation but not lectin pathway activation. The peptides are useful for various therapeutic indications.

In some embodiments, the invention is based on the identification and modification of peptides of 15 amino acids from Polar Assortant (PA) peptide (SEQ ID NO: 2), modifications of the peptides, and methods of their use. The PA peptide is a scrambled peptide derived from human astrovirus protein, called CP1 (SEQ ID NO: 1). The PA peptide is also known as PIC1 (Peptide Inhibitors of Complement C1), AstroFend, AF, or SEQ ID NO: 2. The PIC1 peptide was originally named as such because it was found to be associated with diseases mediated by the complement system. A PEGylated form of the PIC1 peptide, called PA-dPEG24 (SEQ ID NO: 3), has 24 PEG moieties on the C-terminus of the peptide and was shown to have improved effects on complement inhibition. A form of the PIC1 peptide with the amino acid derivative sarcosine at position 8, called PA-I8Sar (SEQ ID NO: 4), also has improved effects on complement inhibition. A form of the PIC1 peptide with the amino acid derivative sarcosine at position 9, called PA-C9Sar (SEQ ID NO: 5), also has improved effects on complement inhibition. PA-dPEG24, PA-I8Sar, and PA-C9Sar are described in, e.g., U.S. Pat. No. 10,005,818, and U.S. Patent Publication No. US2019/0209660. As used herein, the term “PIC1 peptides” include SEQ ID NOs: 6-35 and 54-55 which are stapled forms of SEQ ID NO: 4 and/or substitutions of SEQ ID NO: 4 that have one or more D-enantiomeric forms of amino acids in place of the usual L-enantiomers, and SEQ ID NOs: 36-53 which are stapled forms of SEQ ID NO: 5 and/or substitutions of SEQ ID NO: 5 that have one or more D-enantiomeric forms of amino acids in place of the usual L-enantiomers.

In some aspects, the invention is directed to peptides that are stapled forms of PA-I8Sar and/or comprise one or more D-enantiomeric amino acid substitutions in the sequence of PA-I8Sar that are able to regulate the classical and lectin pathway activation by binding to C1q and MBL. In some aspects, the invention is directed to peptides that are stapled forms of PA-I9Sar and/or comprise one or more D-enantiomeric amino acid substitutions in the sequence of PA-I9Sar that are able to regulate the classical and lectin pathway activation by binding to C1q and MBL.

In some embodiments, the peptide sequence has at least about 95%, at least about 96%, at least about 97%, at least about 98%, or at least about 99% sequence identity to SEQ ID NOs: 6-55. In some embodiments, the peptide sequence has at least about 95%, at least about 96%, at least about 97%, at least about 98%, or at least about 99% sequence identity to SEQ ID NOs: 6-35 and 54-55. In some embodiments, the peptide sequence has at least about 95%, at least about 96%, at least about 97%, at least about 98%, or at least about 99% sequence identity to SEQ ID NOs: 36-53.

In one aspect, the invention provides a synthetic peptide comprising at least about 95% sequence identity to an amino acid sequence of SEQ ID NO: 6-35 and 54-55. In some embodiments, the invention is a synthetic peptide comprising the amino acid sequence and modifications of SEQ ID NO: 6-35 and 54-55. In one aspect, the invention is a synthetic peptide comprising at least about 95% sequence identity to an amino acid sequence of SEQ ID NO: 36-53. In some embodiments, the invention is a synthetic peptide comprising the amino acid sequence and modifications of SEQ ID NO: 36-53.

In another aspect, the invention provides a combination of peptides disclosed herein. In some embodiments, the invention provides a composition comprising at least one synthetic peptide selected from the group consisting of SEQ ID NO: 6-55, and variants thereof. In another aspect, the invention provides a composition comprising at least one synthetic peptide selected from the group consisting of SEQ ID NO: 6-35 and 54-55, and variants thereof. In another aspect, the invention provides a composition comprising at least one synthetic peptide selected from the group consisting of SEQ ID NO: 36-53, and variants thereof. In another aspect, the composition further comprises another D-enantiomeric and/or stapled peptide form of SEQ ID NO: 4 and/or SEQ ID NO: 5, and variants thereof. In another aspect, the composition further comprises one or more of SEQ ID NO: 2, 3, 4, and/or 5, and variants thereof.

In another aspect, the invention is a pharmaceutical composition comprising a therapeutically effective amount of at least one synthetic peptide selected from the group consisting of SEQ ID NO: 6-55, and variants thereof. In another aspect, the invention is a pharmaceutical composition comprising a therapeutically effective amount of at least one synthetic peptide selected from the group consisting of SEQ ID NO: 6-35 and 54-55, and variants thereof. In another aspect, the invention is a pharmaceutical composition comprising a therapeutically effective amount of at least one synthetic peptide selected from the group consisting of SEQ ID NO: 36-53, and variants thereof. In another aspect, the pharmaceutical composition further comprises another D-enantiomeric and/or stapled peptide form of SEQ ID NO: 4 and/or SEQ ID NO: 5, and variants thereof. In another aspect, the pharmaceutical composition further comprises one or more of SEQ ID NO: 2, 3, 4, and/or 5, and variants thereof.

In another aspect, the invention provides a pharmaceutical composition comprising a therapeutically effective amount of at least one synthetic peptide selected from the group consisting of SEQ ID NO: 6-55, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 6-35 and 54-55, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 36-53, and variants thereof, and at least one pharmaceutically acceptable carrier, diluent, or excipient. In another aspect, the pharmaceutical composition further comprises another D-enantiomeric and/or stapled peptide form of SEQ ID NO: 4 and/or SEQ ID NO: 5. In another aspect, the pharmaceutical composition further comprises one or more of SEQ ID NO: 2, 3, 4, and/or 5, and variants thereof.

In one aspect, the invention provides a synthetic peptide comprising at least about 95% sequence identity to an amino acid sequence selected from the group of SEQ ID NO: 6-55.

In some embodiments, the invention provides a synthetic peptide comprising an amino acid sequence selected from the group consisting of SEQ ID NO: 6-55. In some embodiments, the invention provides a synthetic peptide comprising at least about 95% sequence identity to amino acid sequence selected from the group consisting of SEQ ID NOs: 9, 19, 22, 25. In some embodiments, the invention provides a synthetic peptide comprising an amino acid sequence selected from the group consisting of SEQ ID NOs: 9, 19, 22, and 25. In some embodiments, the invention provides a synthetic peptide comprising at least about 95% sequence identity to SEQ ID NO: 9. In some embodiments, the invention provides a synthetic peptide comprising at least about 95% sequence identity to SEQ ID NO: 19. In some embodiments, the invention provides a synthetic peptide comprising at least about 95% sequence identity to SEQ ID NO: 22. In some embodiments, the invention provides a synthetic peptide comprising at least about 95% sequence identity to SEQ ID NO: 25. In another aspect, the invention provides a combination of peptides disclosed herein. In some embodiments, the invention provides a composition comprising at least one synthetic peptide selected from the group consisting of SEQ ID NOs: 9, 19, 22, and 25, and variants thereof. In another aspect, the composition further comprises another D-enantiomeric and/or stapled peptide form of SEQ ID NO: 4 and/or SEQ ID NO: 5, and variants thereof. In another aspect, the composition further comprises one or more of SEQ ID NO: 2, 3, 4, and/or 5, and variants thereof.

In another aspect, the invention is a pharmaceutical composition comprising a therapeutically effective amount of at least one synthetic peptide selected from the group consisting of SEQ ID NOs: 9, 19, 22, and 25, and variants thereof. In another aspect, the pharmaceutical composition further comprises another D-enantiomeric and/or stapled peptide form of SEQ ID NO: 4 and/or SEQ ID NO: 5, and variants thereof. In another aspect, the pharmaceutical composition further comprises one or more of SEQ ID NO: 2, 3, 4, and/or 5, and variants thereof.

In a related aspect, the invention provides a pharmaceutical composition comprising a therapeutically effective amount of any of the synthetic peptides disclosed herein and at least one pharmaceutically acceptable carrier, diluent, or excipient.

In a related aspect, the invention provides a method of regulating the complement system comprising administering a pharmaceutical composition as described herein to a subject in need thereof.

In a related aspect, the invention provides a method of inhibiting myeloperoxidase activity comprising administering a pharmaceutical composition as described herein to a subject in need thereof.

In a related aspect, the invention provides a method of inhibiting NETosis comprising administering a pharmaceutical composition as described herein to a subject in need thereof.

In a related aspect, the invention provides a method of inhibiting oxidant activity comprising administering a pharmaceutical composition as described herein to a subject in need thereof.

In a related aspect, the invention provides a method of inhibiting PD-1 binding to PD-L1 comprising administering a pharmaceutical composition as described herein to a subject in need thereof.

In a related aspect, the invention provides a method of inhibiting T cell exhaustion comprising administering a pharmaceutical composition as described herein to a subject in need thereof.

In a related aspect, the invention provides a method of inhibiting angiogenesis comprising administering a pharmaceutical composition as described herein to a subject in need thereof.

These and other objects, features and advantages of the present invention will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIG. 1A-1B show PIC1 peptides' inhibition of complement activation in an ABO incompatibility assay. FIG. 1A shows modifications of PA-I8Sar and FIG. 1B shows modifications of PA-C9Sar. Inhibition of ABO incompatibility hemolysis in a CH50-type assay. Peptides are at a final concentration of 0.5 mM. Values are represented as a percent of the positive control, which consists of human O sera and AB red blood cells in GVBS⁺⁺ buffer. Data are the means of n=3 independent experiments±SEM.

FIG. 2A-2B show half-maximal binding values for PIC1 peptides binding to C1q. Half-maximal binding concentrations were calculated for (2A) PA-I8Sar and (2B) PA-I9Sar variants from the binding curves. Peptide variant PA-0114 was not analyzed due to lack of material from inefficient synthesis and PA-0116 could not be analyzed due to poor solubility. PA-0130, -0131, -0132 and -0133 could not be analyzed as they were not recognized by the primary polyclonal antibody.

FIG. 3A-3B show half-maximal values for PIC1 peptides inhibition of MPO activity. Half-maximal values were calculated for (3A) PA-I8Sar and (3B) PA-C9Sar variants from the activity curves. PA-0119 could not be calculated because it did not titrate down to a half-maximal value.

FIG. 4A-4B show PIC1 peptide inhibition of oxidant activity in a Total Antioxidant Capacity (TAC) assay. Antioxidant activity is measured in copper reducing equivalents (CRE). (4A) PA-I8Sar and (4B) PA-I9Sar variants were tested over a range of concentrations and the maximal amounts of antioxidant activity is reported for each peptide.

FIG. 5 shows PIC1 peptides' inhibition of free DNA. Free DNA, PicoGreen analysis after neutrophil stimulation with PMA and hydrogen peroxide (H₂O₂) compared with neutrophil only control. PIC1 peptides were added to the sera to a final concentration of 2 mM. The graph shows all peptides inhibited free DNA release by neutrophils as a marker of NETosis. Data are the means of n=5 independent experiments±SEM.

FIG. 6A-6D show C1q binding curves. Binding of increasing concentrations of PA-I8Sar modifications (6A-6B) and PA-I9Sar modifications (6C-6D) to immobilized C1q in an ELISA-type assay. Peptide variants PA-0130, -0131, -0132 and -0133 could not be analyzed as they were not recognized by the primary polyclonal antibody. PA-0114 was not analyzed due to lack of material from inefficient synthesis and PA-0116 could not be analyzed due to poor solubility.

FIG. 7A-7D show MPO inhibition curves. Inhibition of MPO activity by increasing concentrations of PA-I8Sar modifications (7A-7B) and PA-I9Sar modifications (7C-7D) in an ELISA-type assay. PA-0119 could not be calculated because it did not titrate down to a half-maximal value.

FIG. 8A-8D show total antioxidant capacity binding curves. Increasing concentrations of PA-I8Sar modifications (8A-8B) and PA-I9Sar modifications (8C-8D) were analyzed for total antioxidant activity. Antioxidant activity is measured in copper reducing equivalents (CRE).

FIG. 9 shows the results of a pharmacokinetic assay for increasing dosages of PA-0117 based on C1q target acquisition (left bar, 400 mg/kg; middle bar, 200 mg/kg, right bar 20 mg/kg).

FIG. 10A-10B show the results of a hemolysis assay and a pharmacokinetic assay for increasing dosages of PA-0127 (left bar, 400 mg/kg; middle bar, 200 mg/kg, right bar 20 mg/kg).

FIG. 11A-11B show the results of a hemolysis assay and a pharmacokinetic assay for increasing dosages of PA-0130 (left bar, 400 mg/kg; middle bar, 200 mg/kg, right bar 20 mg/kg).

FIG. 12 shows the results of a hemolysis assay for increasing dosages of PA-0133 (left bar, 400 mg/kg; middle bar, 200 mg/kg, right bar 20 mg/kg).

FIG. 13 shows inhibition of PD-1 binding to PD-L1 in an ELISA plate-based assay. PIC1 peptides were allowed to bind to immobilized PD-L1 on the surface of the plate. Biotinylated PD-1 was then added and bound PD-1 detected by streptavidin-HRP reagent followed by TMB as the substrate for the colorimetric assay.

FIG. 14 shows that PIC1 peptides RLS-0117, RLS-0118, RLS-0127*, and RLS-0133* were able to inhibit PD-1/PD-L1 mediated cell signaling. PD-1 effector cells were incubated with PD1-L1 aAPC cells in the absence or presence of increasing concentrations of anti-PD-1 antibody (positive control) and select PIC1 peptides. Luminescence was detected in luminometer plate reader. For sake of clarity, PIC1 peptides that did not inhibit signaling are not shown, but are summarized in Table 4.

FIG. 15 shows RLS-0117 binding to CTLA-4, PD-1 and PD-L1 in an ELISA plate-based assay. RLS-0117 was allowed to bind to immobilized CTLA-4, PD-1, PD-L1 on the surface of the plate. Bound C1q served as a positive and control for peptide binding. Increasing amounts of peptides were added to the plates followed by a rabbit polyclonal antibody that recognizes the peptides and then a secondary anti-rabbit antibody conjugated with HRP. The plate was then developed by addition of TMB as the substrate for the colorimetric assay.

FIG. 16 shows that PIC1 peptides RLS-0127*, RLS-0130, RLS-0156, RLS-0170 and RLS-0174 were able to inhibit CTLA-4 mediated cell signaling. CTLA-4 effector cells were incubated with aAPC/Raji Cells in the absence or presence of increasing concentrations of anti-CTLA-4 antibody (positive control), RLS-0127*, RLS-0130, RLS-0156, RLS-0170 and RLS-0174. Luminescence was detected in luminometer plate reader. For sake of clarity, other PIC1 peptides that did not inhibit CTLA-4 function are summarized in Table 4.

FIG. 17 shows that select PIC1 peptides were able to inhibit T-cell exhaustion to varying degrees as measured by reduced levels of apoptotic cell markers Caspase 3/7. Purified human pan-T-cells were subject to stimulation with Dynabeads every 48 hours over the period of 8 days with cells also receiving PIC1 peptides (2 mg/ml) at each stimulation. Cells not receiving Dynabeads were run in parallel to assess background levels of Caspase 3/7 signal. At the eighth day, cells were harvested and the levels of Caspase 3/7 determined by ELISA.

FIG. 18A-18F show that select PIC1 peptides were able to reverse T-cell exhaustion to varying degrees as measured by increased levels of cytokines IL-2 (18A-18C) and IFN-gamma (18D-18F). Purified human pan-T-cells were subject to stimulation with Dynabeads every 48 hours over the period of 8 days with cells also receiving PIC1 peptides (2 mg/ml) at each stimulation. Cell supernatants were collected at each stimulation and levels of IL-2 and IFN-gamma assayed by ELISA.

FIG. 19A-19C show PIC1 peptides binding to VEGF in an ELISA plate-based assay. PIC1 peptides were allowed to bind to immobilized VEGF on the surface of the plate. A fixed amount of PIC1 peptides (1 mg/ml) were added to the plates followed by a rabbit polyclonal antibody that recognizes the peptides and then a secondary anti-rabbit antibody conjugated with HRP. The plate was then developed by addition of TMB as the substrate for the colorimetric assay.

FIG. 20 shows that specific PIC1 peptides were able to inhibit non-VEGF mediated angiogenesis induced by LPS. HUVEC cells were incubated with the indicated PIC1 peptides followed by LPS addition and plating onto extracellular matrix. After overnight incubation, evidence of angiogenesis was determined by fluorescence microscopy. Cells receiving LPS and not receiving LPS served as positive and negative controls for angiogenesis.

FIG. 21A-21C show that RLS-0127* and RLS-0133* both inhibit complement activation and MPO peroxidase activity and that RLS-0127* also binds C1q. (21A) RLS-0127* and RLS-0133* were evaluated for complement inhibition in an ABO hemolysis assay using human O plasma. Increasing amounts of each peptide were added to the assay and hemolysis was evaluated by absorbance at 450 nm in a plate reader. (21B) RLS-0127* and RLS-0133* were evaluated for MPO peroxidase inhibition in a plate-based assay. Increasing amounts of each peptide were added to wells containing bound, purified human MPO and reduction in peroxidase activity by the TMB substrate was evaluated by absorbance at 450 nm in a plate reader. (21C) RLS-0127* and RLS-0133* were evaluated for C1q binding in a plate-based assay. Increasing amounts of each peptide were added to wells containing bound, purified human C1q which was then probed with antibody to the peptides followed by secondary antibody-HRP conjugate. Development with TMB substrate was evaluated by absorbance at 450 nm in a plate reader. RLS-0133* is not recognized by the rabbit polyclonal antibody and thus binding to C1q could not be determined. RLS-0088 was used as a positive control for all assays.

### DETAILED DESCRIPTION OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to synthetic peptides that are stapled and/or include one or more D-enantiomeric forms of the amino acids.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

As used herein, the term “combination” of a synthetic peptide according to the claimed invention and at least a second pharmaceutically active ingredient means at least two, but any desired combination of compounds can be delivered simultaneously or sequentially (e.g., within a 24 hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “analog” or “functional analog” refers to a related modified form of a polypeptide, wherein at least one amino acid substitution, deletion, or addition has been made such that said analog retains substantially the same biological activity as the unmodified form, in vivo and/or in vitro.

The terms “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

Several different computer programs are available to determine the degree of identity between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at www.accelrys.com/products/gcg), using either a Blosum 62 matrix or a PAM250 matrix, and a gap weight of 16, 14, 12, 10, 8, 6, or 4 and a length weight of 1, 2, 3, 4, 5, or 6. These different parameters will yield slightly different results but the overall percentage identity of two sequences is not significantly altered when using different algorithms.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

“Sequence identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95%, e.g., at least 96%, 97%, 98%, 99%, or 100% “sequence identity” to a reference nucleotide sequence, it is intended that the nucleotide sequence of the given polynucleotide is identical to the reference sequence except that the given polynucleotide sequence may include up to 5, 4, 3, 2, 1, or 0 point mutations per each 100 nucleotides of the reference nucleotide sequence. In other words, in a polynucleotide having a nucleotide sequence having at least 95%, e.g., at least 96%, 97%, 98%, 99%, or 100% sequence identity relative to the reference nucleotide sequence, up to 5%, 4%, 3%, 2%, 1%, or 0% of the nucleotides in the reference sequence may be deleted or substituted with another nucleotide, or a number of nucleotides up to 5%, 4%, 3%, 2%, 1%, or 0% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95%, e.g., at least 96%, 97%, 98%, 99%, or 100% sequence identity to a reference amino acid sequence, it is intended that the given amino acid sequence of the polypeptide is identical to the reference sequence except that the given polypeptide sequence may include up to 5, 4, 3, 2, 1, or 0 amino acid alterations per each 100 amino acids of the reference amino acid sequence. In other words, to obtain a given polypeptide sequence having at least 95%, e.g., at least 96%, 97%, 98%, 99%, or 100% sequence identity with a reference amino acid sequence, up to 5%, 4%, 3%, 2%, 1%, or 0% of the amino acid residues in the reference sequence may be deleted or substituted with another amino acid, or a number of amino acids up to 5%, 4%, 3%, 2%, 1%, or 0% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

As used herein, the term “immune response” includes innate immune responses as well as T-cell mediated and/or B-cell mediated immune responses. Exemplary immune responses include T cell responses, e.g., cytokine production and cellular cytotoxicity, and B cell responses, e.g., antibody production. In addition, the term “immune response” includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes (e.g., neutrophils).

“Parenteral” administration of an immunogenic composition includes, e.g., subcutaneous (s.c.), intravenous (i.v.), intramuscular (i.m.), or intradermal (i.d.) injection, or infusion techniques.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

A “variant” of a polypeptide according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein. As used herein, the term “variant” includes peptides with at least about 95% identity to the peptides disclosed herein.

Within the meaning of the present invention, the term “conjoint administration” is used to refer to administration of a composition according to the invention and another therapeutic agent simultaneously in one composition, or simultaneously in different compositions, or sequentially (preferably, within a 24 hour period).

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Peptide Compositions of the Invention

Modifications of the amino acid structure of CP1 has led to the discovery of additional peptides that are able to regulate complement activation, such as C1q activity. It was previously demonstrated that substitution of isoleucine with sarcosine at position 8 (IALILEP(Sar)CCQERAA, SEQ ID NO: 4, PA-I8Sar) and position 9 (IALILEPI(Sar)CQERAA, SEQ ID NO: 5, PA-C9Sar) resulted in peptides with increased solubility without PEGylation and enhanced inhibition of biological activity compared to the parent molecule (IALILEPICCQERAA-dPEG24; SEQ ID NO: 3, PA-dPEG24) in in vitro assays of classical complement pathway activation/inhibition, myeloperoxidase (MPO) inhibition, oxidant and NET activity. To determine if more potent peptides could be identified, amino acid variants based on the PA-I8Sar and PA-C9Sar backbone were synthesized and consisted of stapled peptides or peptides with D-amino acids individually substituted at each position in the PA-I8Sar and PA-C9Sar peptide sequence (Table 1). Four peptides based on the PA-I8Sar backbone contained a combination of a staple and D-amino acid combinations. Without wishing to be bound by theory, stapling technology can increase peptide stability and enhance biological activity by locking the peptide molecule into a bioactive α-helix secondary structure. Without wishing to be bound by theory, D-amino acid substitutions may impart additional stability to peptides, increasing their in vivo half-life. All but one of these peptides were readily soluble in water and were evaluated for biological activity in the various in vitro assays.

The term “peptide(s),” as used herein, refers to amino acid sequences, which may be naturally occurring, or peptide mimetics, peptide analogs and/or synthetic derivatives (such as for example and not limitation, stapled peptides, sarcosine substitutions, D-amino acid substitutions, and PEGylated peptides) of about 15 amino acids based on SEQ ID NO: 4 or SEQ ID NO: 5. In addition, the peptide may be less than about 15 amino acid residues, such as between about 10 and about 15 amino acid residues and such as peptides between about 5 to about 10 amino acid residues. Peptide residues of, for example, 5, 6, 7, 8, 9, 10, 11, 12, 13, 14, and 15 amino acids are equally likely to be peptides within the context of the present invention. Peptides can also be more than 15 amino acids, such as, for example, 16, 17, 18, 19, and 20, or more amino acids.

The disclosed peptides are generally constrained (that is, have some element of structure as, for example, the presence of amino acids that initiate a β turn or β pleated sheet, or, for example, are cyclized by the presence of disulfide bonded Cys residues) or unconstrained (that is, linear) amino acid sequences of about 15 amino acid residues, or less than about 15 amino acid residues.

Substitutes for an amino acid within the peptide sequence may be selected from other members of the class to which the amino acid belongs. For example, the nonpolar (hydrophobic) amino acids include alanine, leucine, isoleucine, valine, proline, phenylalanine, tryptophan, and methionine. Amino acids containing aromatic ring structures include phenylalanine, tryptophan, and tyrosine. The polar neutral amino acids include glycine, serine, threonine, cysteine, tyrosine, asparagine, and glutamine. The positively charged (basic) amino acids include arginine and lysine. The negatively charged (acidic) amino acids include aspartic acid and glutamic acid. For example, one or more amino acid residues within the sequence can be substituted by another amino acid of a similar polarity, which acts as a functional equivalent, resulting in a silent alteration.

A conservative change generally leads to less change in the structure and function of the resulting protein. A non-conservative change is more likely to alter the structure, activity, or function of the resulting protein. For example, the peptide of the present disclosure comprises one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

Particularly preferred amino acid substitutions include:
a) Ala for Glu or vice versa, such that a negative charge may be reduced;
b) Lys for Arg or vice versa, such that a positive charge may be maintained;
c) Ala for Arg or vice versa, such that a positive charge may be reduced;
d) Glu for Asp or vice versa, such that a negative charge may be maintained;
e) Ser for Thr or vice versa, such that a free —OH can be maintained;
f) Gln for Asn or vice versa, such that a free NH2 can be maintained;
g) Ile for Leu or for Val or vice versa, as roughly equivalent hydrophobic amino acids;
h) Phe for Tyr or vice versa, as roughly equivalent aromatic amino acids; and
i) Ala for Cys or vice versa, such that disulfide bonding is affected.

Substitutes for an amino acid within the peptide sequence may be selected from any amino acids, including, but not limited to alanine, arginine, asparagine, aspartic acid, cysteine, glutamic acid, glutamine, glycine, histidine, isoleucine, leucine, lysine, methionine, phenylalanine, proline, pyrolysine, selenocysteine, serine, threonine, tryptophan, tyrosine, valine, N-formyl-L-methionine, sarcosine, or other N-methylated amino acids. In some embodiments, sarcosine substitutes for an amino acid within the peptide sequence.

Stapling of the peptides can be achieved by using non-native amino acids in desired positions in the peptide that have side chains that can be linked, e.g., by covalent bonding, in order to introduce alpha-helices into the peptide structure. See, e.g., Ali et al., Stapled Peptides Inhibitors: A New Window for Target Drug Discovery, Comput Struct Biotechnol J. 2019; 17: 263-281 and Walensky et al., Hydrocarbon-Stapled Peptides: Principles, Practice, and Progress, J Med Chem. 2014 Aug. 14; 57(15): 6275-6288.

In one embodiment, the invention discloses synthetic peptides derived from human astrovirus coat protein, the peptides comprising the amino acid sequences and modifications of SEQ ID NOs: 6-55. In some embodiments, the invention discloses synthetic peptides derived from human astrovirus coat protein, the peptides comprising the amino acid sequences and modifications of SEQ ID NOs: 6-55 as shown in Table 1 below. The stapled amino acids are indicated by underlining and the D-enantiomeric amino acids are indicated in bold.

TABLE 1
List of Peptides of the Invention.

| SEQ | | |
|---|---|---|
| ID | | |
| NO. | Sequence | Description |
| 1 | PAICQRATATLGTVG | Astrovirus CP protein |
| | SNTSGTTEIEACILL | |
| 2 | IALILEPICCQERAA | PA (PIC1) |
| 3 | IALILEPICCQERAA-PEG24 | PA-dPEG24 |
| 4 | IALILEP(Sar)CCQERAA | PA-18Sar (RLS-0088) |
| 5 | IALILEPI(Sar)CQERAA | PA-C9Sar (RLS-0089) |
| 6 | IALILR8P(Sar)CCQES5AA | PA-0114/RLS-0114, stapled modification of SEQ ID NO: 4 |
| 7 | IR8LILEP(Sar)S5CQERAA | PA-0115/RLS-0115, stapled modification of SEQ ID NO: 4 |
| 8 | IALILEP(Sar)CCS5ERAS5 | PA-0116/RLS-0116, stapled modification of SEQ ID NO: 4 |
| 9 | IALILEP(Sar)CS5QERS5A | PA-0117/RLS-0117, stapled modification of SEQ ID NO: 4 |
| 10 | IALILEP(Sar)S5CQES5AA | PA-0118/RLS-0118, stapled modification of SEQ ID NO: 4 |
| 11 | IALILS5P(Sar)CS5QERAA | PA-0119/RLS-0119, stapled modification of SEQ ID NO: 4 |
| 12 | dIALILEP(Sar)CCQERAA | PA-0120/RLS-0120, D-enantiomer substitution of SEQ ID |
| | | NO: 4 |
| 13 | IdALILEP(Sar)CCQERAA | PA-0121/RLS-0121, D-enantiomer substitution of SEQ ID |
| | | NO: 4 |
| 14 | IALdILEP(Sar)CCQERAA | PA-0123/RLS-0123, D-enantiomer substitution of SEQ ID |
| | | NO: 4 |
| 15 | IALIdLEP(Sar)CCQERAA | PA-0124/RLS-0124, D-enantiomer substitution of SEQ ID |
| | | NO: 4 |
| 16 | IALILdEP(Sar)CCQERAA | PA-0125/RLS-0125, D-enantiomer substitution of SEQ ID |
| | | NO: 4 |
| 17 | IALILEdP(Sar)CCQERAA | PA-0126/RLS-0126, D-enantiomer substitution of SEQ ID |
| | | NO: 4 |
| 18 | IALILEP(Sar)dCCQERAA | PA-0127/RLS-0127, D-enantiomer substitution of SEQ ID |
| | | NO: 4 |
| 19 | IALILEP(Sar)CdCQERAA | PA-0128/RLS-0128, D-enantiomer substitution of SEQ ID |
| | | NO: 4 |
| 20 | IALILEP(Sar)CCdQERAA | PA-0129/RLS-0129, D-enantiomer substitution of SEQ ID |
| | | NO: 4 |
| 21 | IALILEP(Sar)CCQdERAA | PA-0130/RLS-0130, D-enantiomer substitution of SEQ ID |
| | | NO: 4 |
| 22 | IALILEP(Sar)CCQEdRAA | PA-0131/RLS-0131, D-enantiomer substitution of SEQ ID |
| | | NO: 4 |
| 23 | IALILEP(Sar)CCQERdAA | PA-0132/RLS-0132, D-enantiomer substitution of SEQ ID |
| | | NO: 4 |
| 24 | IALILEP(Sar)CCQERAdA | PA-0133/RLS-0134, D-enantiomer substitution of SEQ ID |
| | | NO: 4 |
| 25 | IALR8LEP(Sar)CCS5ERAA | PA-0135/RLS-0135, stapled modification of SEQ ID NO: 4 |
| 26 | IALIR8EP(Sar)CCQS5RAA | PA-0136/RLS-0136, stapled modification of SEQ ID NO: 4 |
| 27 | IALILER8(Sar)CCQERAS5 | PA-0137/RLS-0137, stapled modification of SEQ ID NO: 4 |
| 28 | S5ALIS5EP(Sar)CCQERAA | PA-0138/RLS-0138, stapled modification of SEQ ID NO: 4 |
| 29 | IAS5ILES5(Sar)CCQERAA | PA-0139/RLS-0139, stapled modification of SEQ ID NO: 4 |
| 30 | IALIS5EP(Sar)S5CQERAA | PA-0140/RLS-0140, stapled modification of SEQ ID NO: 4 |
| 31 | IALILES5(Sar)CCS5ERAA | PA-0141/RLS-0141, stapled modification of SEQ ID NO: 4 |
| 32 | IALILEP(Sar)dCS5QERS5A | PA-0143/RLS-0142, stapled and D-enantiomeric |
| | | modification of SEQ ID NO: 4 |
| 33 | IALILEP(Sar)CS5QdERS5A | PA-0144/RLS-0144, stapled and D-enantiomeric |
| | | modification of SEQ ID NO: 4 |
| 34 | IALILEP(Sar)CS5QERS5dA | PA-0145/RLS-0145, stapled and D-enantiomeric |
| | | modification of SEQ ID NO: 4 |
| 35 | R8ALILEPS5(Sar)CQERAA | PA-0146/RLS-0146, stapled modification of SEQ ID NO: 4 |
| 36 | IALR8LEPI(Sar)CS5ERAA | PA-0147/RLS-0147, stapled modification of SEQ ID NO: 5 |
| 37 | IALIR8EPI(Sar)CQS5RAA | PA-0148/RLS-0148, stapled modification of SEQ ID NO: 5 |
| 38 | IALILR8PI(Sar)CQES5AA | PA-0149/RLS-0149, stapled modification of SEQ ID NO: 5 |
| 39 | S5ALIS5EPI(Sar)CQERAA | PA-0151/RLS-0151, stapled modification of SEQ ID NO: 5 |
| 40 | IAS5ILES5I(Sar)CQERAA | PA-0153/RLS-0153, stapled modification of SEQ ID NO: 5 |
| 41 | IALILEPI(Sar)CS5ERAS5 | PA-0155/RLS-0155, stapled modification of SEQ ID NO: 5 |
| 42 | dIALILEPI(Sar)CQERAA | PA-0156/RLS-0156, D-enantiomeric modification of SEQ |
| | | ID NO: 5 |
| 43 | IdALILEPI(Sar)CQERAA | PA-0157/RLS-0157, D-enantiomeric modification of SEQ |
| | | ID NO: 5 |
| 44 | IAdLILEPI(Sar)CQERAA | PA-0158/RLS-0158, D-enantiomeric modification of SEQ |
| | | ID NO: 5 |
| 45 | IALdILEPI(Sar)CQERAA | PA-0159/RLS-0159, D-enantiomeric modification of SEQ |
| | | ID NO: 5 |
| 46 | IALIdLEPI(Sar)CQERAA | PA-0160/RLS-0160, D-enantiomeric modification of SEQ |
| | | ID NO: 5 |
| 47 | IALILdEPI(Sar)CQERAA | PA-0161/RLS-0161, D-enantiomeric modification of SEQ |
| | | ID NO: 5 |
| 48 | IALILEdPI(Sar)CQERAA | PA-0162/RLS-0162, D-enantiomeric modification of SEQ |
| | | ID NO: 5 |
| 49 | IALILEPdI(Sar)CQERAA | PA-0163/RLS-0163, D-enantiomeric modification of SEQ |
| | | ID NO: 5 |
| 50 | IALILEPI(Sar)CdQERAA | PA-0165/RLS-0165, D-enantiomeric modification of SEQ |
| | | ID NO: 5 |
| 51 | IALILEPI(Sar)CQdERAA | PA-0166/RLS-0166, D-enantiomeric modification of SEQ |
| | | ID NO: 5 |
| 52 | IALILEPI(Sar)CQEdRAA | PA-0167/RLS-0167, D-enantiomeric modification of SEQ |
| | | ID NO: 5 |
| 53 | IALILEPI(Sar)CQERAdA | PA-0169/RLS-0169, D-enantiomeric modification of SEQ |
| | | ID NO: 5 |
| 54 | IALILEP(Sar)dCCQERRA | PA-0127*, D-enantiomer substitution of SEQ ID NO: 4 |
| | | with an additional mutation |
| 55 | IALILEP(Sar)CCQERA | PA-0133*, D-enantiomer substitution of SEQ ID NO: 4 |
| | | with an additional mutation |

In some embodiments, the peptide sequence has at least about 95%, at least about 96%, at least about 97%, at least about 98%, or at least about 99% sequence identity to SEQ ID NOs: 6-55. In some embodiments, the peptide sequence has at least about 95%, at least about 96%, at least about 97%, at least about 98%, or at least about 99% sequence identity to SEQ ID NOs: 6-35 and 54-55. In some embodiments, the peptide sequence has at least about 95%, at least about 96%, at least about 97%, at least about 98%, or at least about 99% sequence identity to SEQ ID NOs: 36-53.

In one aspect, the invention is a synthetic peptide comprising the amino acid sequence and modifications of SEQ ID NO: 6-55. In some embodiments, the invention is a synthetic peptide comprising the amino acid sequence and modifications of SEQ ID NO: 6-35 and 54-55. In some embodiments, the invention is a synthetic peptide comprising the amino acid sequence and modifications of SEQ ID NO: 36-53.

In another aspect, the invention provides a combination of peptides disclosed herein. In some embodiments, the invention provides a composition comprising at least one synthetic peptide selected from the group consisting of SEQ ID NO: 6-55, and variants thereof. In another aspect, the invention provides a composition comprising at least one synthetic peptide selected from the group consisting of SEQ ID NO: 6-35 and 54-55, and variants thereof. In another aspect, the invention provides a composition comprising at least one synthetic peptide selected from the group consisting of SEQ ID NO: 36-53, and variants thereof. In another aspect, the composition further comprises another D-enantiomeric and/or stapled peptide form of SEQ ID NO: 4 and/or SEQ ID NO: 5, and variants thereof. In another aspect, the composition further comprises one or more of SEQ ID NO: 2, 3, 4, and/or 5, and variants thereof.

In another aspect, the invention is a pharmaceutical composition comprising a therapeutically effective amount of at least one synthetic peptide selected from the group consisting of SEQ ID NO: 6-55, and variants thereof. In another aspect, the invention is a pharmaceutical composition comprising a therapeutically effective amount of at least one synthetic peptide selected from the group consisting of SEQ ID NO: 6-35 and 54-55, and variants thereof. In another aspect, the invention is a pharmaceutical composition comprising a therapeutically effective amount of at least one synthetic peptide selected from the group consisting of SEQ ID NO: 36-53, and variants thereof. In another aspect, the pharmaceutical composition further comprises another D-enantiomeric and/or stapled peptide form of SEQ ID NO: 4 and/or SEQ ID NO: 5, and variants thereof. In another aspect, the pharmaceutical composition further comprises one or more of SEQ ID NO: 2, 3, 4, and/or 5, and variants thereof.

In another aspect, the invention provides a pharmaceutical composition comprising a therapeutically effective amount of at least one synthetic peptide selected from the group consisting of SEQ ID NO: 6-55, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 6-35 and 54-55, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 36-53, and variants thereof, and at least one pharmaceutically acceptable carrier, diluent, or excipient. In another aspect, the pharmaceutical composition further comprises another D-enantiomeric and/or stapled peptide form of SEQ ID NO: 4 and/or SEQ ID NO: 5. In another aspect, the pharmaceutical composition further comprises one or more of SEQ ID NO: 2, 3, 4, and/or 5, and variants thereof.

In one aspect, the invention provides a synthetic peptide comprising at least about 95% sequence identity to an amino acid sequence selected from the group of SEQ ID NO: 6-55.

In some embodiments, the invention provides a synthetic peptide comprising an amino acid sequence selected from the group consisting of SEQ ID NO: 6-55. In some embodiments, the invention provides a synthetic peptide comprising at least about 95% sequence identity to amino acid sequence selected from the group consisting of SEQ ID NOs: 9, 19, 22, 25. In some embodiments, the invention provides a synthetic peptide comprising an amino acid sequence selected from the group consisting of SEQ ID NOs: 9, 19, 22, and 25. In some embodiments, the invention provides a synthetic peptide comprising at least about 95% sequence identity to SEQ ID NO: 9. In some embodiments, the invention provides a synthetic peptide comprising at least about 95% sequence identity to SEQ ID NO: 19. In some embodiments, the invention provides a synthetic peptide comprising at least about 95% sequence identity to SEQ ID NO: 22. In some embodiments, the invention provides a synthetic peptide comprising at least about 95% sequence identity to SEQ ID NO: 25. In another aspect, the invention provides a composition comprising at least one synthetic peptide selected from the group consisting of SEQ ID NOs: 9, 19, 22, and 25, and variants thereof. In another aspect, the composition further comprises another D-enantiomeric and/or stapled peptide form of SEQ ID NO: 4 and/or SEQ ID NO: 5, and variants thereof. In another aspect, the composition further comprises one or more of SEQ ID NO: 2, 3, 4, and/or 5, and variants thereof.

In another aspect, the invention is a pharmaceutical composition comprising a therapeutically effective amount of at least one synthetic peptide selected from the group consisting of SEQ ID NOs: 9, 19, 22, and 25, and variants thereof. In another aspect, the pharmaceutical composition further comprises another D-enantiomeric and/or stapled peptide form of SEQ ID NO: 4 and/or SEQ ID NO: 5, and variants thereof. In another aspect, the pharmaceutical composition further comprises one or more of SEQ ID NO: 2, 3, 4, and/or 5, and variants thereof.

The disclosed peptides can selectively regulate C1q and MBL activation without affecting alternative pathway activity and are, thus, ideal for preventing and treating diseases mediated by the dysregulated activation of the classical and lectin pathways, respectively. Specific blockade of classical and lectin pathways are particularly needed, as both of these pathways have been implicated in ischemia-reperfusion induced injury in many animal models. [Castellano et al., “Therapeutic targeting of classical and lectin pathways of complement protects from ischemia-reperfusion-induced renal damage.” Am J Pathol. 2010; 176(4):1648-59; Lee et al., “Early complement factors in the local tissue immunocomplex generated during intestinal ischemia/reperfusion injury.” Mol. Immunol. 2010 February; 47(5):972-81; Tjernberg, et al., “Acute antibody-mediated complement activation mediates lysis of pancreatic islets cells and may cause tissue loss in clinical islet transplantation.” Transplantation. 2008 Apr. 27; 85(8):1193-9; Zhang et al. “The role of natural IgM in myocardial ischemia-reperfusion injury.” J Mol Cell Cardiol. 2006 July; 41(1):62-7). The alternative pathway is essential for immune surveillance against invading pathogens, and humans with alternative pathway defects suffer severe bacterial infections. By binding and inactivating C1q and MBL, the peptides can efficiently regulate classical and lectin pathway activation while leaving the alternative pathway intact.

The term “regulate,” as used herein, refers to i) controlling, reducing, inhibiting or regulating the biological function of an enzyme, protein, peptide, factor, byproduct, or derivative thereof, either individually or in complexes; ii) reducing the quantity of a biological protein, peptide, or derivative thereof, either in vivo or in vitro; or iii) interrupting a biological chain of events, cascade, or pathway known to comprise a related series of biological or chemical reactions. The term “regulate” may thus be used, for example, to describe reducing the quantity of a single component of the complement cascade compared to a control sample, reducing the rate or total amount of formation of a component or complex of components, or reducing the overall activity of a complex process or series of biological reactions, leading to such outcomes as cell lysis, formation of convertase enzymes, formation of complement-derived membrane attack complexes, inflammation, or inflammatory disease. In an in vitro assay, the term “regulate” may refer to the measurable change or reduction of some biological or chemical event, but the person of ordinary skill in the art will appreciate that the measurable change or reduction need not be total to be “regulatory.”

In some embodiments, the present invention relates to therapeutically active peptides having the effects of regulating the complement system.

Modulation of C1q Interaction with C1q Receptors

C1q interactions with C1q receptors appear to play important roles in homeostatic functions such as scavenging of apoptotic cellular debris and immune complexes as well as T cell signaling through antigen presenting cells (macrophage and dendritic cells). Currently, no clinical pharmacological agents modulate the interaction of C1q with C1q receptors.

The disclosed peptides can be used to block C1q binding to C1q receptors, including calreticulin/cC1qR. The ability of the disclosed peptides to block binding of C1q to cellular receptors may have an important role in modulating intracellular signaling processes mediated by C1q binding to C1q receptors.

Myeloperoxidase (MPO) Activity

Myeloperoxidase (MPO) is an enzyme from neutrophils that creates hypochlorite (bleach) in acute inflammation and damages invading and host cells alike. This enzyme is known to be destructive to host tissues in many diseases.

In some embodiments, the peptides disclosed herein blocked the enzymatic activity of MPO. In some embodiments, MPO activity present in the lysates of purified human neutrophils can be directly inhibited by the peptides. In some embodiments, the invention demonstrates that the peptides have anti-inflammatory activity.

Hemolysis Inhibition

The peptides of the invention, including SEQ ID NO: 6-56, can block complement-mediated lysis of AB human red blood cells (RBC) by O serum in vitro. This assay mimics ABO incompatibility.

Neutrophil Extracellular Trap Formation

NETs are formed upon stimulation of neutrophils in acute inflammation and may damage host tissue. These NETs consist of extracellular DNA coated with neutrophil derived proteins such as histones, neutrophil elastase and MPO that can be toxic to host cells and tissues. In some embodiments the peptides disclosed herein blocked the formation of NETs.

Oxidant Activity

Oxidant activity resulting in the formation of reactive oxygen species can be formed in acute inflammation leading to host cell and tissue damage. In some embodiments, the peptides disclosed herein possess antioxidant activity against oxidant generating molecules such as MPO.

### Pharmaceutical Compositions of the Invention

The present disclosure provides pharmaceutical compositions capable of regulating the complement system, comprising at least one peptide, as discussed above, and at least one pharmaceutically acceptable carrier, diluent, stabilizer, or excipient. Pharmaceutically acceptable carriers, excipients, or stabilizers are nontoxic to recipients at the dosages and concentrations employed. They can be solid, semi-solid, or liquid. The pharmaceutical compositions of the present invention can be in the form of tablets, pills, powders, lozenges, sachets, cachets, elixirs, suspensions, emulsions, solutions, or syrups.

The pharmaceutical compositions of the present invention are prepared by mixing the peptide(s) having the appropriate degree of purity with pharmaceutically acceptable carriers, diluents, or excipients. Examples of formulations and methods for preparing such formulations are well known in the art. The pharmaceutical compositions of the present invention are useful as a prophylactic and therapeutic agent for various disorders and diseases, as set forth above. In one embodiment, the composition comprises a therapeutically effective amount of at least one peptide disclosed herein. In another embodiment, the composition comprises at least one other active ingredient effective in regulating the complement system. In another embodiment, the composition comprises at least one other active ingredient effective in treating at least one disease associated with the complement system. In another embodiment, the composition comprises at least one other active ingredient effective in treating at least one disease that is not associated with the complement system. The term “therapeutically effective amount,” as used herein, refers to the total amount of each active component that is sufficient to show a benefit to the subject.

The therapeutically effective amount of the peptide(s) varies depending on several factors, such as the condition being treated, the severity of the condition, the time of administration, the route of administration, the rate of excretion of the peptide(s) employed, the duration of treatment, the co-therapy involved, and the age, gender, weight, and condition of the subject, etc. One of ordinary skill in the art can determine the therapeutically effective amount. Accordingly, one of ordinary skill in the art may need to titer the dosage and modify the route of administration to obtain the maximal therapeutic effect.

The effective daily dose generally is within the range of from about 0.001 to about 200 milligrams per kilogram (mg/kg) of body weight, including about 5 to about 160 mg/kg, about 10 to about 160 mg/kg, about 40 mg/kg to about 160 mg/kg, and about 40 mg/kg to about 100 mg/kg. This dose can be achieved through a 1-6 time(s) daily dosing regimen. Alternatively, optimal treatment can be achieved through a sustained release formulation with a less frequent dosing regimen.

In another aspect, the invention is a pharmaceutical composition comprising a therapeutically effective amount of at least one synthetic peptide selected from the group consisting of SEQ ID NO: 6-55, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 6-35 and 54-55, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 36-53, and variants thereof, and at least one pharmaceutically acceptable carrier, diluent, or excipient. In another aspect, the pharmaceutical composition further comprises another D-enantiomeric and/or stapled peptide form of SEQ ID NO: 4 and/or SEQ ID NO: 5, and at least one pharmaceutically acceptable carrier, diluent, or excipient. In another aspect, the pharmaceutical composition further comprises one or more of SEQ ID NO: 2, 3, 4, and/or 5, and variants thereof, and at least one pharmaceutically acceptable carrier, diluent, or excipient.

The compositions of the invention can comprise a carrier and/or excipient. While it is possible to use a peptide of the present invention for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice. Oral formulations readily accommodate additional mixtures, such as, e.g., milk, yogurt, and infant formula. Solid dosage forms for oral administration can also be used and can include, e.g., capsules, tablets, caplets, pills, troches, lozenges, powders, and granules. Non-limiting examples of suitable excipients include, e.g., diluents, buffering agents (e.g., sodium bicarbonate), preservatives, stabilizers, binders, compaction agents, lubricants, dispersion enhancers, disintegration agents, antioxidants, flavoring agents, sweeteners, and coloring agents. Those of relevant skill in the art are well able to prepare suitable solutions.

In one embodiment of any of the compositions of the invention, the composition is formulated for delivery by a route such as, e.g., oral, topical, rectal, mucosal, sublingual, nasal, naso/oro-gastric gavage, parenteral, intraperitoneal, intradermal, transdermal, intrathecal, nasal, and intracheal administration. In one embodiment of any of the compositions of the invention, the composition is in a form of a liquid, foam, cream, spray, powder, or gel. In one embodiment of any of the compositions of the invention, the composition comprises a buffering agent (e.g., sodium bicarbonate).

Administration of the compounds and compositions in the methods of the invention can be accomplished by any method known in the art. Non-limiting examples of useful routes of delivery include oral, rectal, fecal (by enema), and via naso/oro-gastric gavage, as well as parenteral, intraperitoneal, intradermal, transdermal, intrathecal, nasal, and intracheal administration. The active agent may be systemic after administration or may be localized by the use of regional administration, intramural administration, or use of an implant that acts to retain the active dose at the site of implantation.

The useful dosages of the compounds and formulations of the invention can vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses may be effective to achieve a therapeutic effect. While it is possible to use a compound of the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

Solutions or suspensions can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity, such as sodium chloride or dextrose.

In instances in which the agents exhibit insufficient solubility, methods for solubilizing agents may be used. Such methods are known to those of skill in this art, and include, but are not limited to, using co-solvents, such as, e.g., dimethylsulfoxide (DMSO), using surfactants, such as TWEEN® 80, or dissolution in aqueous sodium bicarbonate. Pharmaceutically acceptable derivatives of the agents may also be used in formulating effective pharmaceutical compositions.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art (e.g., Remington's Pharmaceutical Sciences, Mack Publishing Company, Easton, Pa., 15th Edition, 1975). The composition or formulation to be administered will, in any event, contain a quantity of the active agent in an amount sufficient to alleviate the symptoms of the treated subject.

The active agents or pharmaceutically acceptable derivatives may be prepared with carriers that protect the agent against rapid elimination from the body, such as time release formulations or coatings. The compositions may include other active agents to obtain desired combinations of properties.

Parenteral administration, generally characterized by injection, either subcutaneously, intramuscularly or intravenously, is also contemplated herein. Injectables can be prepared in conventional forms, either as liquid solutions or suspensions, solid forms suitable for solution or suspension in liquid prior to injection, or as emulsions. Suitable excipients include, by way of example and without limitation, water, saline, dextrose, glycerol or ethanol. In addition, if desired, the pharmaceutical compositions to be administered may also contain minor amounts of non-toxic auxiliary substances, such as wetting or emulsifying agents, pH buffering agents, stabilizers, solubility enhancers, and other such agents, such as, for example, sodium acetate, sorbitan monolaurate, triethanolamine oleate and cyclodextrins.

Lyophilized powders can be reconstituted for administration as solutions, emulsions, and other mixtures or formulated as solids or gels. The sterile, lyophilized powder is prepared by dissolving an agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution can be apportioned into vials for lyophilization. Each vial can contain, by way of example and without limitation, a single dosage (10-1000 mg, such as 100-500 mg) or multiple dosages of the agent. The lyophilized powder can be stored under appropriate conditions, such as at about 4° C. to room temperature. Reconstitution of this lyophilized powder with water for injection provides a formulation for use in parenteral administration.

### Methods of Use

Another aspect of the invention provides a method of regulating the complement system comprising administering a therapeutically effective amount of the peptides and/or the pharmaceutical compositions of the invention to a subject in need thereof.

In another aspect, the invention provides a method of inhibiting myeloperoxidase activity comprising administering a therapeutically effective amount of the peptides and/or the pharmaceutical compositions of the invention to a subject in need thereof.

In another aspect, the invention provides a method of inhibiting NETosis comprising administering a therapeutically effective amount of the peptides and/or the pharmaceutical compositions of the invention to a subject in need thereof.

In another aspect, the invention provides a method of inhibiting oxidant activity comprising administering a therapeutically effective amount of the peptides and/or the pharmaceutical compositions of the invention to a subject in need thereof.

In another aspect, the invention provides a method of inhibiting PD-1 binding to PD-L1 comprising administering a therapeutically effective amount of the peptides and/or the pharmaceutical compositions of the invention to a subject in need thereof.

In another aspect, the invention provides a method of inhibiting T cell exhaustion comprising administering a therapeutically effective amount of the peptides and/or the pharmaceutical compositions of the invention to a subject in need thereof.

In another aspect, the invention provides a method of inhibiting angiogenesis comprising administering a therapeutically effective amount of the peptides and/or the pharmaceutical compositions of the invention to a subject in need thereof

### Combination Therapies

A further embodiment of the invention provides a method of regulating the complement system, comprising administering to a subject a pharmaceutical composition of the present invention. While the pharmaceutical compositions of the present invention can be administered as the sole active pharmaceutical agent, they can also be used in combination with one or more therapeutic or prophylactic agent(s) that is(are) effective for regulating the complement system. In this aspect, the method of the present invention comprises administrating a pharmaceutical composition of the present invention before, concurrently, and/or after one or more additional therapeutic or prophylactic agents effective in regulating the complement system.

The pharmaceutical compositions of the present invention can be administered with additional agent(s) in combination therapy, either jointly or separately, or by combining the pharmaceutical compositions and the additional agent(s) into one composition. The dosage is administered and adjusted to achieve maximal regulation of the complement system. For example, both the pharmaceutical compositions and the additional agent(s) are usually present at dosage levels of between about 10% and about 150%, more preferably, between about 10% and about 80%, of the dosage normally administered in a mono-therapy regimen.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Characterization of SEQ ID NOs: 6-53

The inventors found that a sarcosine amino acid substitution scan of PA-dPEG24 revealed that substitution of this amino acid at different positions of the peptide, resulted in peptides that were soluble in water in the absence of PEGylation and also displayed increased inhibitory activity in in vitro assays of classical complement pathway activation, MPO activation, NET formation and antioxidant activity [11]. Two variants in which sarcosine replaced isoleucine at position 8 (PA-I8Sar) or cysteine at position 9 (PA-C9Sar) displayed increased inhibitory activity in the respective assays. Using the peptide backbone of both PA-I8Sar and PA-C9Sar, the inventors created peptide peptides with D-amino acid substitutions and/or engineered stapling that display increased potency in the various functional assays and retain aqueous solubility in the absence of PEGylation.

### Materials and Reagents

Peptides were synthesized by New England Peptide (Gardner, Mass.) to >90% purity (Table 1). Stapled peptides were produced through a one-component stapling technique employing S-pentenylalanine (S5) at i,i+4 positions for one-turn stapling or combining either R-octenylalanine (R8)/S-pentenylalanine (S5) at i,i+7 positions. Sequences corresponding to PA-0135, PA-0137, PA-0139, PA-0141 and PA-0146 could not be produced due to an excess of cis isomer over trans isomer during synthesis making purification of the usually favored trans isomer difficult (NEP, personnel correspondence) and were thus not pursued. D-enantiomer forms of each amino acid individually substituted at each position in the PA-I8Sar and PA-C9Sar peptide sequence except for sarcosine at position 8 (PA-I8Sar) and position 9 (PA-C9Sar) as no enantiomeric form exists. With the exception of PA-0116, all peptides were dissolved in water and the pH was adjusted with NaOH. Purified C1q was purchased from Complement Technology (Tyler, Tex.). Purified MPO was purchased from Lee BioSolutions (Maryland Heights, Mo.) and tetramethylbenzidine (TMB) was purchased from Thermo Fisher (Waltham Mass.). Buffers included Complement permissive GVBS⁺⁺ buffer (veronal-buffered saline with 0.1% gelatin, 0.15 mM CaCl₂, and 1 mM MgCl₂ [12]).

### Methods

### Normal Human Serum (NHS)

Blood type O normal human serum (NETS) was prepared as previously described [12]. Briefly, blood from at least 4 healthy human donors was collected in Vacutainer tubes without additives (red top). The blood was incubated for 30 minutes at room temperature and 2 hours on ice to clot and the serum separated. The sera was then pooled, aliquoted and frozen at −80° C.

### Hemolytic Assays of Complement Activity

For hemolytic complement assays, human red blood cells (RBCs) from type AB donors were purified, washed, and standardized to 1.0×10⁹ cells/ml, as previously described [13]. Human sera from type O donors at a 15% final concentration was combined with 0.5 mM of the peptides and the volume was brought up to 0.2 ml with GVBS⁺⁺ and 5.0×10⁷ RBCs. The samples were incubated for 1 hour at 37° C. and then spun at 3,000 rpm for 5 minutes and the supernatant was collected and read at 412 nm. Values are represented as a percent of the positive control, which consists of human O sera and AB red blood cells in GVBS⁺⁺ buffer.

### C1q Binding Assay

The C1q binding assay was performed as previously described [11]. Briefly, an Immunlon-2 HB ELISA plate was coated with 1 μg/ml C1q in bicarbonate buffer overnight at 4° C. The plates were washed with PBS-T (phosphate buffered saline+0.1% Tween) and then blocked with 1% gelatin/PBS for 2 hours at room temperature. After washing, the plates were incubated with the peptides starting at 2.5 mg/ml and then serially diluted in 1% gelatin/PBS for 1 hour at room temperature followed by washing. Plates were then probed with rabbit antibody raised against the lead peptide IALILEPICCQERAA (SEQ ID NO: 2) that lacked PEGylation [11] at 1:1000 in 1% gelatin/PBS for 1 hour at room temperature followed by a goat anti-rabbit HRP (Sigma Aldrich, St Louis, Mo.) at 1:1,000 in 1% gelatin/PBS for 1 hour at room temperature with a washing step in between. After addition of TMB substrate solution to the wells, the reaction was stopped using 1N H₂SO₄, and the plate read on a BioTek Synergy HT plate reader at 450 nm.

### MPO Activity Assay

The MPO activity assay was performed as previously described [10]. Briefly, peptides were diluted to 12 mg/ml and serially titrated in a 96 well plate at a volume of 0.02 ml. MPO was diluted to 20 μg/ml and 0.02 ml was added to the titrated peptides. TMB (3,30,5,50-tetramethylbenzidine) (0.1 ml) was added to each well for 2 minutes, followed by 0.1 ml of 2.5 N H₂SO₄ for another 2 minutes, and then read on a 96 well plate reader (BioTek) at 450 nm.

### Total Antioxidant Capacity Assay

As previously reported [6], the TAC (Total Antioxidant Capacity) Assay (Cell Biolabs, Inc, San Diego, Calif.) was used to measure the antioxidant capacity of the PIC1 variants based on the reduction of copper (II) to copper (I). The kit protocol was performed per the manufacturer's recommendations.

### NETosis Assay

Free DNA as a marker for NET formation was measured by PicoGreen as previously described [11]. Briefly, 2.0×10⁶ human neutrophils in 96-well plates were stimulated with 12 nM PMA and 0.05% H₂O₂ in RPMI with or without the indicated peptide variants (2 mM) for 2.5 hr in a 37° C. humified incubator supplemented with 5% CO₂, allowing NETosis to occur. Neutrophils in RPMI alone served as a negative control. Fifty units of monococcal nuclease (Fisher) were added to each well to allow for digestion of released extracellular DNA for 10 min at 37° C. The preparation was then aliquoted into an adjacent well and mixed 1:1 with prepared PICO green reagent (Fisher). The fluorescence was then quantified on a BioTek microplate reader at excitation 485 nm/emission 528 nm.

### Statistical Analysis

Quantitative data were analyzed determining means, standard error (SEM), and Student's t-test [14] using Excel (Microsoft, Redmond, Wash.).

### Results

### Peptides

It was previously demonstrated that substitution of isoleucine with sarcosine at position 8 (IALILEP(Sar)CCQERAA, SEQ ID NO:4, PA-I8Sar) and position 9 (IALILEPI(Sar)CQERAA, SEQ ID NO: 5, PA-C9Sar) resulted in peptides with increased solubility without PEGylation and enhanced inhibition of biological activity compared to the parent molecule (IALILEPICCQERAA-dPEG24, SEQ ID NO:3) in in vitro assays of classical complement pathway, MPO, oxidant and NET activity [11]. To determine if more potent peptides could be identified, amino acid variants based on the PA-I8Sar and PA-C9Sar backbone were synthesized and consisted of stapled peptides or peptides with D-amino acids individually substituted at each position in the PA-I8Sar and PA-C9Sar peptide sequence (Tables 1 and 2, respectively). Three peptides based on the PA-I8Sar backbone contained a combination of a staple and D-amino acid combinations (PA-0143 to PA-0145). Stapling technology has been demonstrated to increase peptide stability and enhance biological activity by locking the peptide molecule into a bioactive α-helix secondary structure [2] whereas D-amino acid substitutions can impart additional stability to natural peptides increasing their in vivo half-life [15]. Each of these peptides, with the exception of PA-0116, were readily soluble in water and were evaluated for biological activity in the various in vitro assays.

### Complement Inhibition and C1q Binding

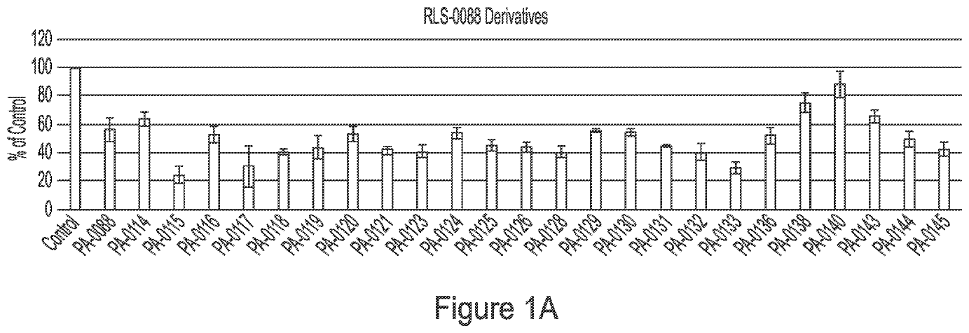

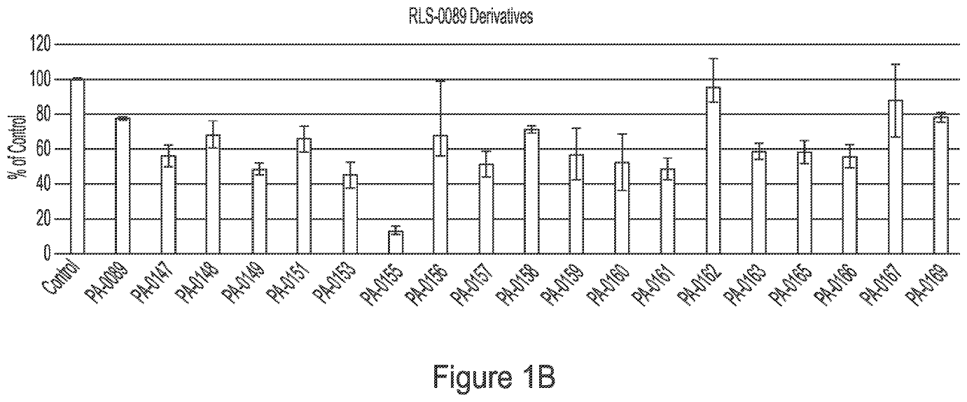

To assess the extent to which the peptide variants inhibit antibody-initiated complement activation, an ABO incompatibility ex vivo assay was utilized in which purified erythrocytes from a ‘type AB+’ donor are incubated with sera from a ‘type O’ subject containing anti-A and anti-B antibodies [13]. Peptides were tested at a concentration of 0.8 mg/ml (˜0.5 mM). For peptides based on the PA-I8Sar backbone, peptides PA-0114 through PA-0133, which consisted of stapled peptides (PA-0114 through PA-0119) and D-amino acid substitutions (PA-0120 through PA-0133) inhibited ABO incompatible hemolysis to a similar or greater extent compared to the PA-I8Sar control (FIG. 1A). Compared to the PA-I8Sar variant, stapled peptides PA-0115 dramatically and unexpectedly decreased ABO hemolysis 30% (P=0.06) whereas PA-0117 decreased ABO hemolysis by 25% (P value=0.279). D-amino acid variants PA-0127 and PA-0133 decreased ABO hemolysis by 20% (P value=0.083) and 25% (P value=0.076), respectively.

Additional stapled peptides based on the PA-I8Sar backbone were designed (PA-0135 through PA-0141 and PA-0146), however only PA-0136, -0138 and -0140 could be synthesized in sufficient amounts for analysis (Table 2). PA-0136 inhibited complement to the same level as the parent molecule, PA-I8Sar, whereas PA-0134, -0138, and -0140 inhibited complement to a lesser extent (FIG. 1A). Given the superior inhibition of hemolytic activity of the stapled peptide PA-0117 and the D-amino acid substitutions PA-0127, -0130 and -0133, peptides combining the PA-0117 with each of these D-amino acid variants (PA-0143 through PA-0145) were synthesized and tested for complement inhibition (FIG. 1A). These peptides ranged from less complement inhibitory activity to approximately a 10% increase in complement inhibition for PA-0145 compared to PA-I8Sar suggesting that combination of the stapled peptide with the D-amino acid substitutions did not display synergistic complement inhibitory activity (FIG. 1A).

Stapled and D-amino acid variants of the PA-C9Sar molecule were next tested for inhibition of complement in the hemolytic assay. The majority of peptides inhibited complement activity slightly more than PA-C9Sar except for variants PA-0162 and -0167 which inhibited complement activity slightly less well; PA-0169 had similar activity to the parent molecule (FIG. 1B). In contrast, stapled peptide PA-0155 unexpectedly decreased ABO hemolysis significantly by 64% (P value<0.001)

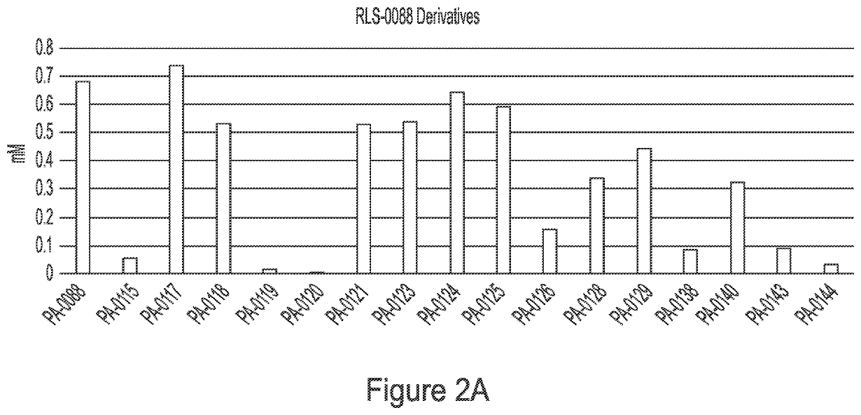

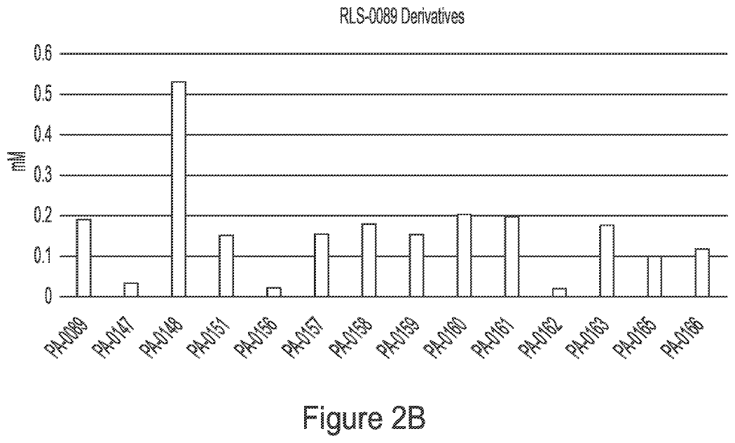

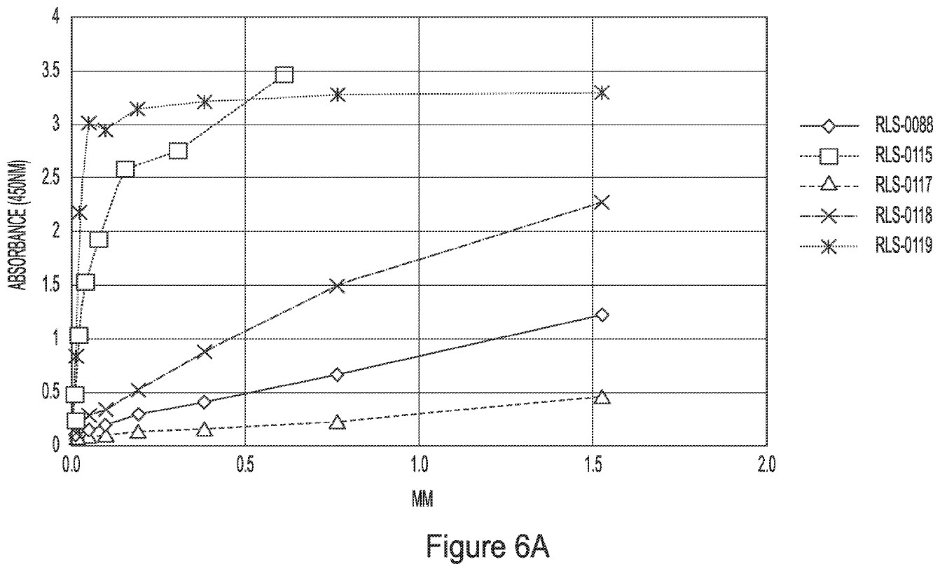

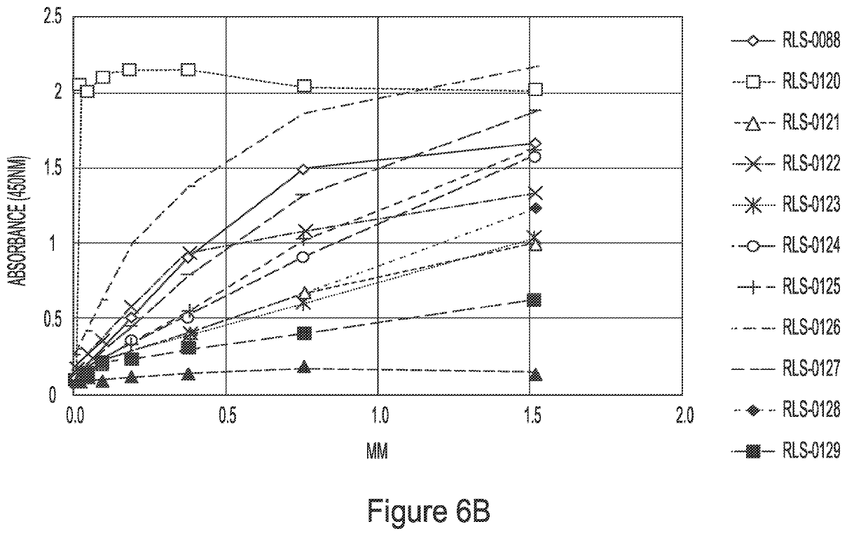

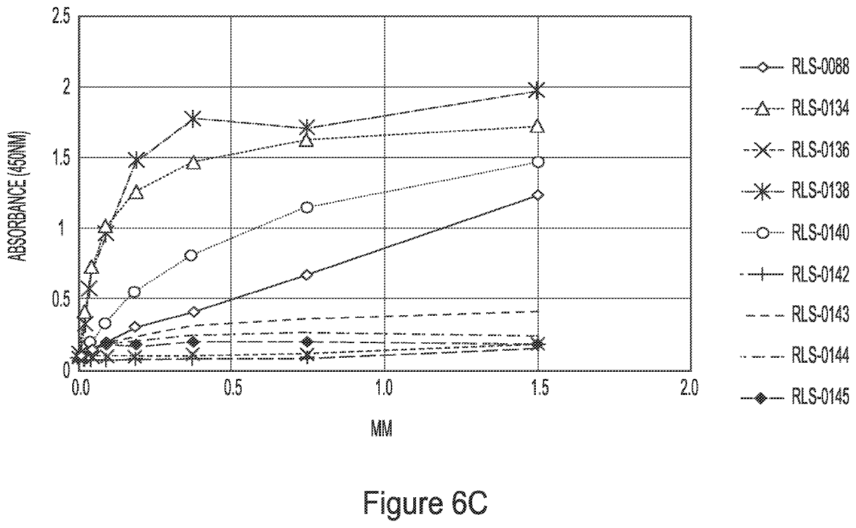

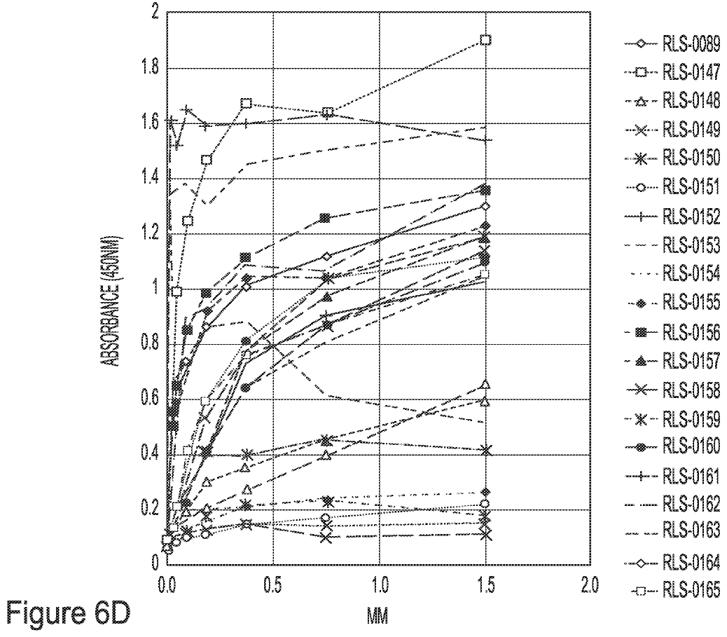

The astrovirus capsid protein and the PIC1 molecules derived from it inhibit classical complement pathway activation by binding to the pattern recognition molecule C1q [3,10,11]. The inventors next tested peptide variant binding to C1q in an ELISA-type assay in which C1q is used as the capture substrate and bound peptide detected with a rabbit polyclonal antibody against the peptide portion of PA-dPEG24 (IALILEPICCQERAA-d24; SEQ ID NO: 3) [11]. Binding curves were derived for the stapled and D-amino acid peptides based on the PA-I8Sar and PA-C9Sar (FIG. 6A-6D) backbone, from which half-maximal binding concentrations were calculated (FIGS. 2A and B, respectively). For the stapled peptides based on the PA-I8Sar backbone, these binding curves and half-maximal binding calculations demonstrate that PA-0115 and PA-0119 had markedly increased binding to C1q compared with PA-I8Sar and PA-0117 and -0118. For the D-amino acid variants, all peptides in which C1q binding was detected, had variable but increased binding to C1q compared to PA-I8Sar (FIG. 2A). For the peptides based on the PA-C9Sar backbone, with the exception of PA-0148, C1q was bound to the same degree or greater than the parent peptide (FIG. 2B). Surprisingly, while a number of peptides showed superior complement inhibition and C1q binding activity such as PA-0115 (compare FIGS. 1A and 2A), the strength of binding to C1q did not strictly correlate with inhibition of classical pathway complement activation of other peptide variants (e.g., PA-0162) (compare FIGS. 1B and 2B) suggesting that complement inhibitory activity may not be wholly dictated by strength of binding to C1q.

### Myeloperoxidase Inhibition

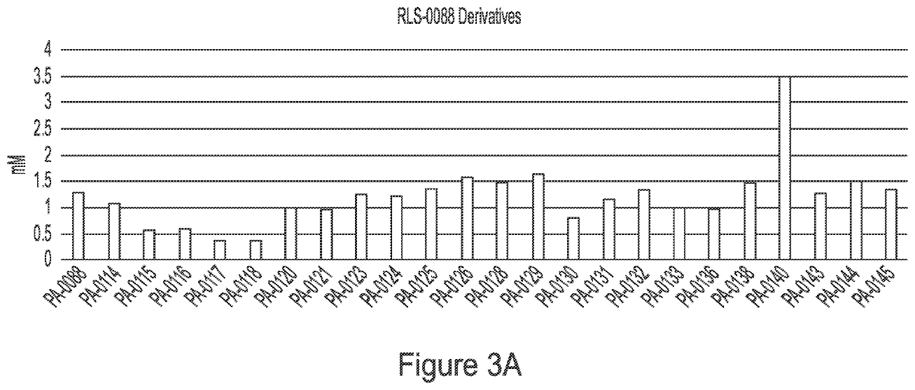

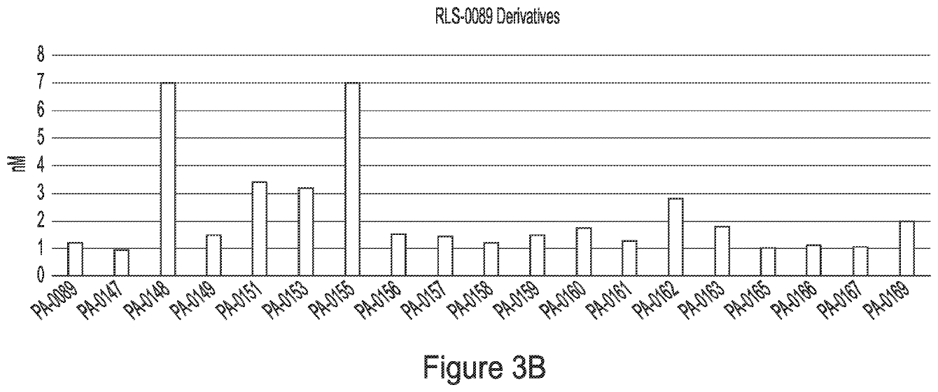

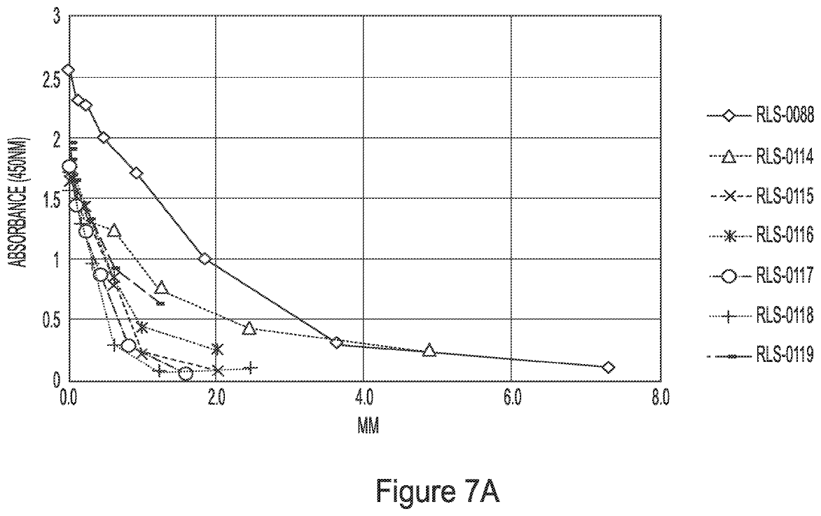

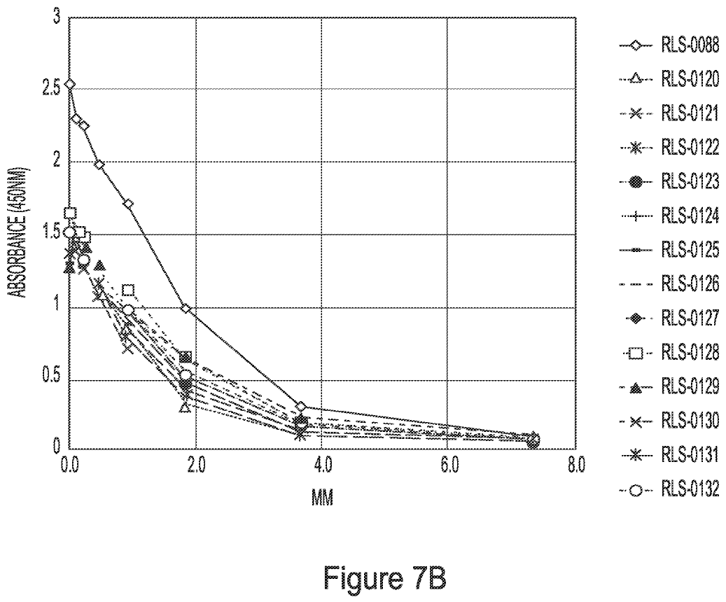

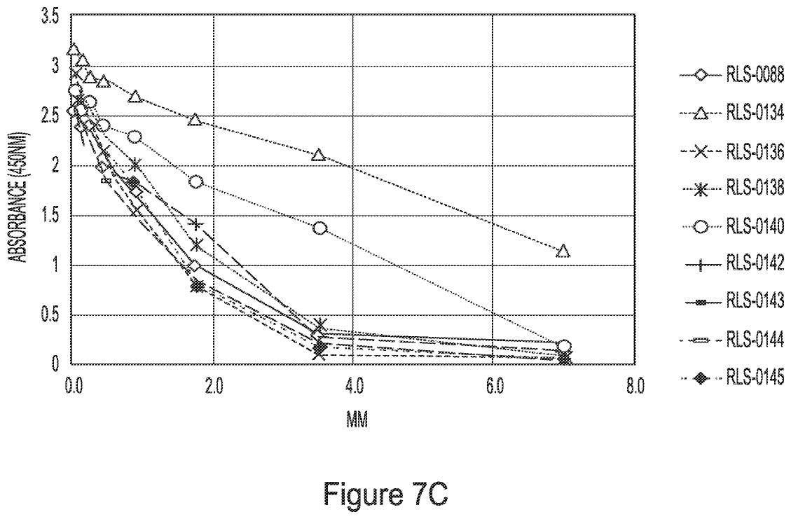

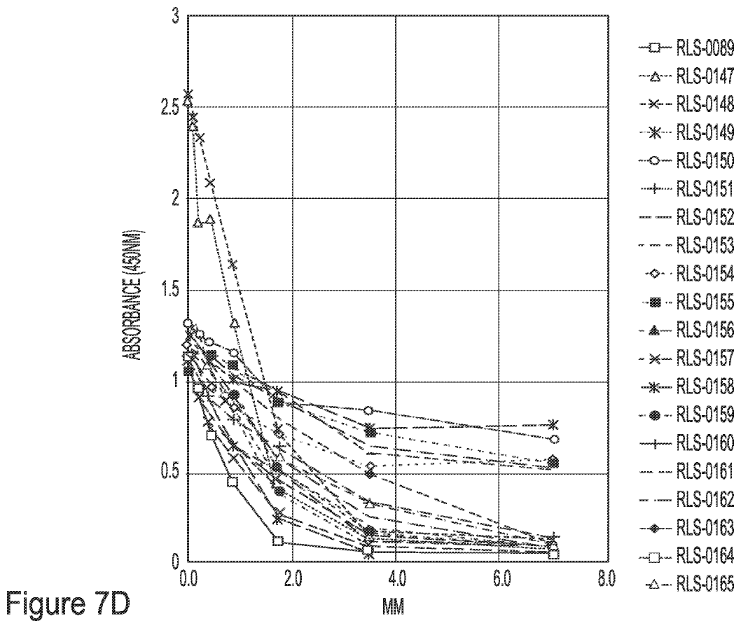

The inventors have previously demonstrated that PA-dPEG24 and sarcosine substitutions can bind myeloperoxidase (MPO) and inhibit the activity of this enzyme [4, 11]. To ascertain the inhibition of MPO activity by the various peptides, a range of concentrations of the stapled peptides and D-amino acid variants were tested (FIG. 7A-7D) and half-maximal activity levels were calculated from the dose-response curves (FIGS. 3A and B, respectively). For the PA-I8Sar peptides, most demonstrated similar levels of MPO inhibition or slightly improved MPO inhibition with the exception of stapled peptides PA-0140 which had reduced inhibition of MPO activity (FIG. 3A). For the PA-C9Sar peptides, the majority of the variants maintained similar inhibitory activity as the parent PA-C9Sar peptide with the exception of stapled peptides PA-0148, -0151, -0153, -0155, and -0163 which demonstrated decreased MPO inhibition (FIG. 3B). Thus, for both the PA-I8Sar and PA-C9Sar variants, some of the stapled peptides demonstrated varying effects on MPO binding affinity whereas the D-amino acid substitutions did not dramatically alter MPO binding.

### Antioxidant Capacity

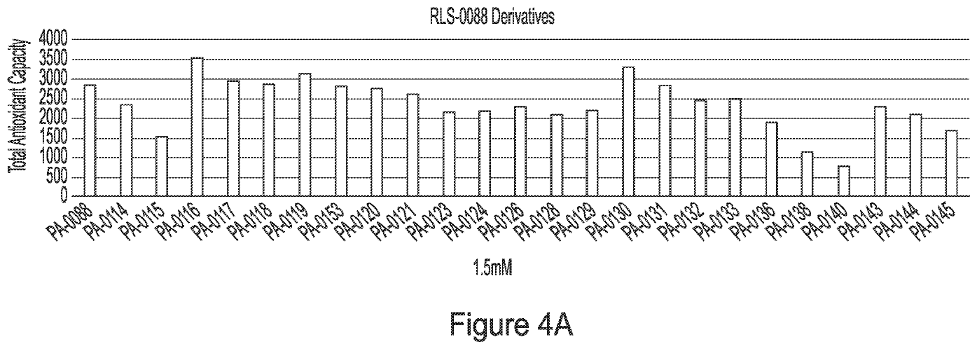

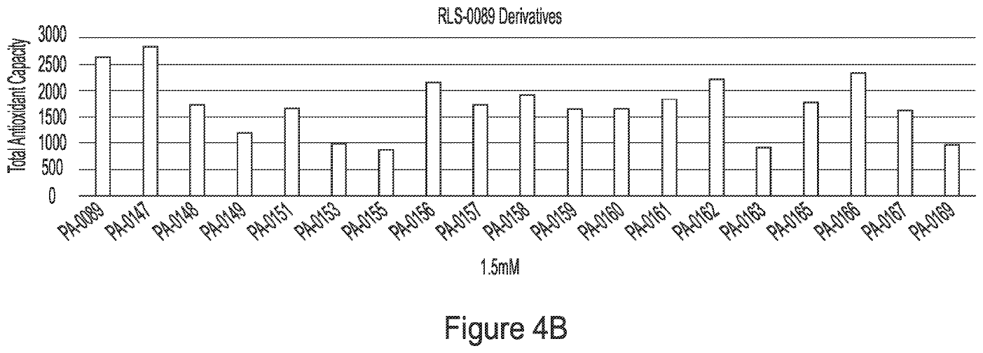

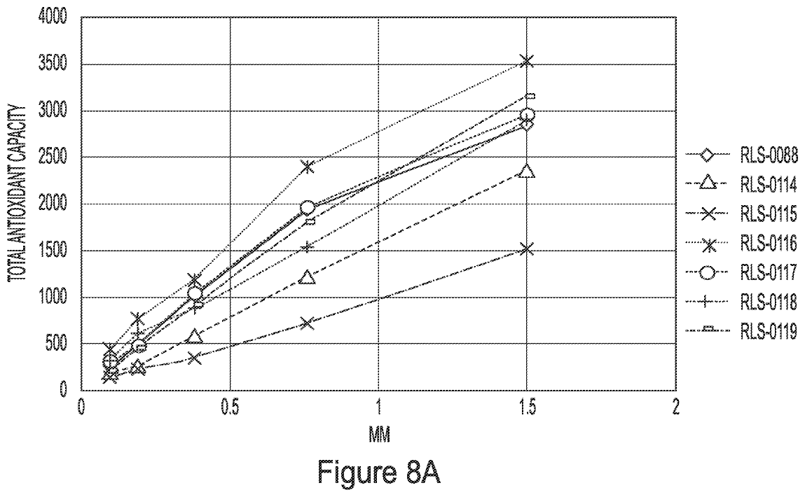

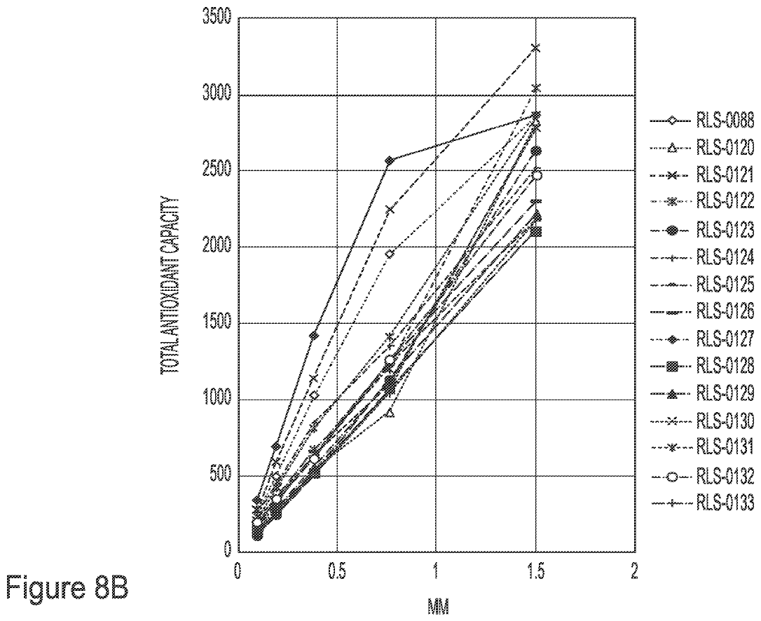

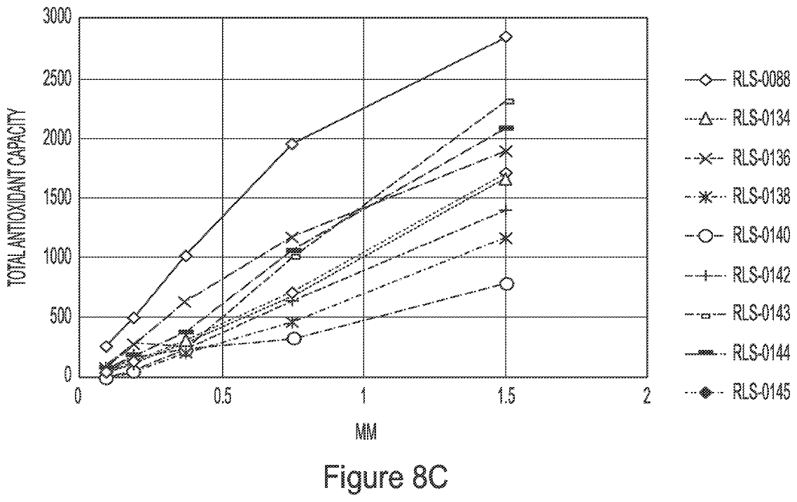

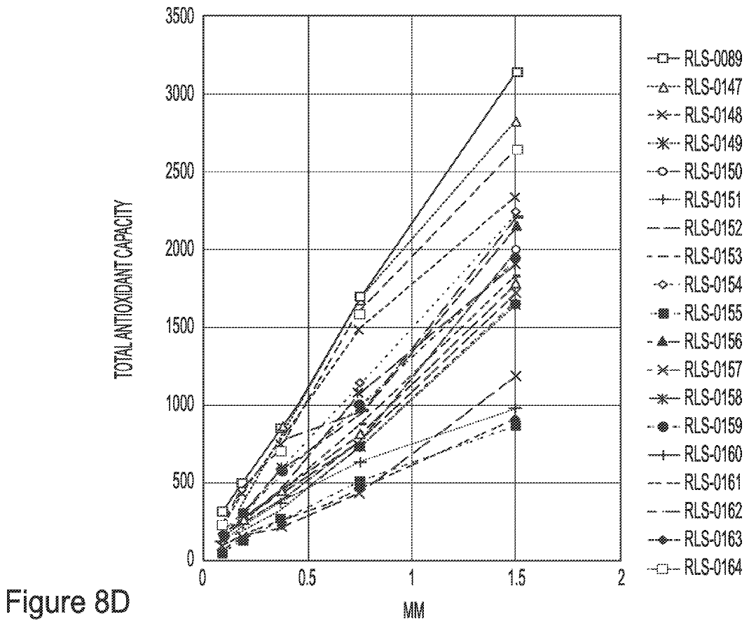

The antioxidant properties for the PIC1 variants were evaluated in a Total Antioxidant Capacity (TAC) assay, as previously reported [6]. Total antioxidant activity over a range of peptide concentrations were determined for the stapled and the D-amino acid peptides based on the PA-I8Sar and PA-C9Sar backbones (FIG. 8A-8D), with the activity of the highest peptide concentration reported (1.5 mM) (FIGS. 4A and B, respectively). For the stapled peptides based on the parent PA-I8Sar peptide, PA-0114 and -0015 showed a reduction in total antioxidant capacity whereas PA-0116 had increased activity and PA-0117 through PA-0119 maintained the same amount of activity as the parent peptide. For the D-amino acid variants, there was variation in the amount of antioxidant activity with the peptides having similar, slightly lower and slightly higher levels of total antioxidant capacity. In contrast, stapled peptides (PA-0135 through -0140) as well as combination of stapled and D-amino acid peptides had reduced total antioxidant capacity (FIG. 4A). Surprisingly, the majority of peptides based on the PA-C9Sar peptide showed a reduction in total antioxidant capacity with the exception of stapled peptide PA-0147 which maintained similar activity (FIG. 4B). The inventors have previously demonstrated that both vicinal cysteine residues at positions 9 and 10 of the parent PA-dPEG24 peptide (IALILEPICCQERAA-dPEG24, SEQ ID NO: 3) are essential for antioxidant activity with oxidation of both residues inhibiting this function [6]. These data suggest that the cysteine at position 10 is sufficient to maintain antioxidant activity.

### Free DNA (NETosis) Inhibition

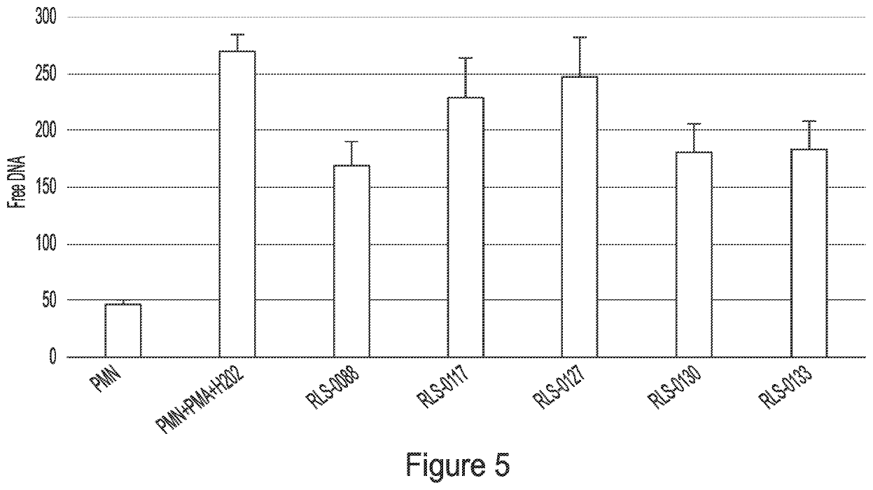

PA-I8Sar and PA-C9Sar have been previously shown to inhibit formation of neutrophil extracellular traps (NETs) by fluorescence microscopy and free DNA as a marker of NETosis [7]. Selected modifications of PA-I8Sar consisting of a stapled peptide (PA-0117) and 3 D-amino acid modifications (PA-0127, -0130 and -0133) were screened for reduction of free DNA by stimulated neutrophils (FIG. 5). As expected, the control (PA-I8Sar) reduced the level of free DNA compared to stimulated neutrophils. PA-0117 and -0127 did not reduce levels of free DNA whereas PA-0130 and -0133 had a reduction in free DNA similar to that of the PA-I8Sar control.

Discussion

The inventors had previously demonstrated that sarcosine substitution of the parental, 15 residue, PEGylated PIC1 molecule (PA-dPEG24) yielded six peptides that were aqueous soluble without PEGylation and had enhanced activity in functional assays of complement, MPO, NETosis and oxidant activity [11]. PA-I8Sar (isoleucine at position 8 substituted for sarcosine) was chosen for further modification by peptide stapling and substitution of D-amino acids to determine if its functional activity in the various assays could be further enhanced. The inventors also performed the same analysis with PA-C9Sar (cysteine at position 9 substituted for sarcosine). While PA-C9Sar did not show as great an enhancement of activity in the various assays compared to PA-I8Sar [11], the inventors were interested to analyze its function in the context of stapling and D-amino acid substitution to see if the single cysteine residue could maintain functional activity. As the inventors have previously demonstrated, both cysteine residues at positions 9 and 10 are critical for the functional activity of the PIC1 molecule [11]. The activity of the variants of PA-I8Sar and PA-C9Sar in the various assays are summarized in Tables 2 and 3.

TABLE 2
PA-I8Sar peptides and summary of properties.

| | SEQ | | | | | |
|---|---|---|---|---|---|---|
| | ID | | ABO | C1q | MPO | TAC |
| Name | NO. | Sequence | Hemolysis | Binding | Activity | Activity |
| PA-0088 | 4 | H2N-IALILEP(Sar)CCQERAA-OH | + | + | + | + |
| PA-0114 | 6 | H2N-IALILR8P(Sar)CCQES5AA-OH | + | ND | ++ | + |
| PA-0115 | 7 | H2N-IR8LILEP(Sar)S5CQERAA-OH | ++++ | ++++ | +++ | + |
| PA-0116 | 8 | H2N-IALILEP(Sar)CCS5ERAS5-OH | + | ND | +++ | +++ |
| PA-0117 | 9 | H2N-IALILEP(Sar)CS5QERS5A-OH | +++ | + | ++++ | ++ |
| PA-0118 | 10 | H2N-IALILEP(Sar)S5CQES5AA-OH | ++ | ++ | ++++ | ++ |
| PA-0119 | 11 | H2N-IALILS5P(Sar)CS5QERAA-OH | ++ | ++++ | ND | +++ |
| PA-0120 | 12 | H2N-dIALILEP(Sar)CCQERAA-OH | + | ++++ | ++ | + |
| PA-0121 | 13 | H2N-IdALILEP(Sar)CCQERAA-OH | ++ | ++ | ++ | + |
| PA-0123 | 14 | H2N-IALdILEP(Sar)CCQERAA-OH | ++ | ++ | + | + |
| PA-0124 | 15 | H2N-IALIdLEP(Sar)CCQERAA-OH | + | ++ | + | + |
| PA-0125 | 16 | H2N-IALILdEP(Sar)CCQERAA-OH | + | ++ | + | + |
| PA-0126 | 17 | H2N-IALILEdP(Sar)CCQERAA-OH | + | +++ | + | + |
| PA-0127 | 18 | H2N-IALILEP(Sar)dCCQERAA-OH | +++ | ++ | ++ | + |
| PA-0128 | 19 | H2N-IALILEP(Sar)CdCQERAA-OH | ++ | ++ | + | + |
| PA-0129 | 20 | H2N-IALILEP(Sar)CCdQERAA-OH | + | ++ | + | + |
| PA-0130 | 21 | H2N-IALILEP(Sar)CCQdERAA-OH | + | ND | ++ | ++++ |
| PA-0131 | 22 | H2N-IALILEP(Sar)CCQEdRAA-OH | ++ | ND | + | + |
| PA-0132 | 23 | H2N-IALILEP(Sar)CCQERdAA-OH | ++ | ND | + | + |
| PA-0133 | 24 | H2N-IALILEP(Sar)CCQERAdA-OH | +++ | ND | ++ | + |
| PA-0135 | 25 | H2N-IALR8LEP(Sar)CCS5ERAA-OH | NA | NA | NA | NA |
| PA-0136 | 26 | H2N-IALIR8EP(Sar)CCQS5RAA-OH | + | ND | ++ | + |
| PA-0137 | 27 | H2N-IALILER8(Sar)CCQERAS5-OH | NA | NA | NA | NA |
| PA-0138 | 28 | H2N-S5ALIS5EP(Sar)CCQERAA-OH | + | ++++ | + | + |
| PA-0139 | 29 | H2N-IAS5ILES5(Sar)CCQERAA-OH | NA | NA | NA | NA |
| PA-0140 | 30 | H2N-IALIS5EP(Sar)S5CQERAA-OH | + | +++ | + | + |
| PA-0141 | 31 | H2N-IALILES5(Sar)CCS5ERAA-OH | NA | NA | NA | NA |
| PA-0143 | 32 | H2N-IALILEP(Sar)dCS5QERS5A-OH | + | ++++ | + | + |
| PA-0144 | 33 | H2N-IALILEP(Sar)CS5QdERS5A-OH | + | ++++ | + | + |
| PA-0145 | 34 | H2N-IALILEP(Sar)CS5QERS5dA-OH | ++ | ND | + | + |
| PA-0146 | 35 | H2N-R8ALILEPS5(Sar)CQERAA-OH | NA | NA | NA | NA |

¹ND: not determined. Peptide sequences PA-0130, -0131, -0132, -0133, -0136, and -0145 had minimal binding to C1q. PA-0114 and -0116 were not analyzed due to peptide availability and reduced solubility in water, respectively. PA-0119 MPO half-maximal activity could not be determined as the peptide did not titrate.
²NA: not analyzed. Peptide sequences PA-0135, -137, -139, -141 and -146 could not be efficiently synthesized.

TABLE 3
PA-C9Sar peptides and summary of properties.

| | SEQ | | | | | |
|---|---|---|---|---|---|---|
| | ID | | ABO | C1q | MPO | TAC |
| Name | NO. | Sequence | Hemolysis | Binding | Activity | Activity |
| PA-0089 | 5 | H2N-IALILEPI(Sar)CQERAA-OH | + | + | + | + |
| PA-0147 | 36 | H2N-IALR8LEPI(Sar)CS5ERAA-OH | ++ | ++++ | ++ | ++ |
| PA-0148 | 37 | H2N-IALIR8EPI(Sar)CQS5RAA-OH | + | + | + | + |
| PA-0149 | 38 | H2N-IALILR8PI(Sar)CQES5AA-OH | ++ | ND | + | + |
| PA-0151 | 39 | H2N-S5ALIS5EPI(Sar)CQERAA-OH | + | + | + | + |
| PA-0153 | 40 | H2N-IAS5ILES5I(Sar)CQERAA-OH | ++ | ND | + | + |
| PA-0155 | 41 | H2N-IALILEPI(Sar)CS5ERAS5-OH | ++++ | ND | + | + |
| PA-0156 | 42 | H2N-dIALILEPI(Sar)CQERAA-OH | + | ++++ | + | + |
| PA-0157 | 43 | H2N-IdALILEPI(Sar)CQERAA-OH | ++ | + | + | + |
| PA-0158 | 44 | H2N-IAdLILEPI(Sar)CQERAA-OH | + | + | + | + |
| PA-0159 | 45 | H2N-IALdILEPI(Sar)CQERAA-OH | ++ | + | + | + |
| PA-0160 | 46 | H2N-IALIdLEPI(Sar)CQERAA-OH | ++ | + | + | + |
| PA-0161 | 47 | H2N-IALILdEPI(Sar)CQERAA-OH | ++ | + | + | + |
| PA-0162 | 48 | H2N-IALILEdPI(Sar)CQERAA-OH | + | ++++ | + | + |
| PA-0163 | 49 | H2N-IALILEPdI(Sar)CQERAA-OH | ++ | + | + | + |
| PA-0165 | 50 | H2N-IALILEPI(Sar)CdQERAA-OH | ++ | +++ | + | + |
| PA-0166 | 51 | H2N-IALILEPI(Sar)CQdERAA-OH | ++ | +++ | ++ | + |
| PA-0167 | 52 | H2N-IALILEPI(Sar)CQEdRAA-OH | + | ND | ++ | + |
| PA-0169 | 53 | H2N-IALILEPI(Sar)CQERAdA-OH | ++ | ND | + | + |

¹ND: not determined. For C1q binding, peptide sequence PA-0153 did not titrate and PA-0149, -0155, -0167, and -0169 were not recognized by the polyclonal antibody to the parent peptide sequence, IALILEPICCQERAA-dPEG24 (SEQ ID NO: 3).

PA-I8Sar Peptides. Stapling of the PA-I8Sar molecule at various positions resulted in a subset of peptides that surprisingly had greatly enhanced activity over the parent molecule (PA-0115, -0116, -0117 and -0118). Unexpectedly, some modifications showed differential modulation of functional activity. For example, whereas PA-0115 demonstrated enhanced activity compared to PA-I8Sar in hemolysis, C1q binding and MPO assays, it had much less antioxidant activity. In contrast, PA-0116 showed superior antioxidant activity but similar activity to PA-I8Sar in the hemolytic assay. Other stapled peptides did not appear to enhance functional activity compared to PA-I8 Sar.

As with the stapled peptides, D-amino acid substitutions at each position of the PA-I8Sar molecule, with the exception of sarcosine at position 8, resulted in molecules (PA-0121, -0127, -0128, -0130 and -0133) with varying degrees of enhanced activity in the various assays. Interestingly, with the exception of and -0130, the D-amino acid variants did not demonstrate enhanced anti-oxidant activity. Additionally, combination of PA-0117 with the superior D-amino acid modifications showed no activity enhancement (PA-0143 through -0145).

A subset of the PA-I8Sar peptides were also testing for reduction of free DNA, a biomarker for NETosis as previously reported [11]. The stapled peptide PA-0117 and the D-amino acid variant PA-0127 did not show a reduction in free DNA, but two other D-amino acid variant did demonstrate an ability to inhibit NET formation (PA-0130 and -0133).

PA-C9Sar Peptides. The same strategy of stapling and D-amino acid substitution on the PA-C9Sar backbone unexpectedly yielded peptides with varying levels of functional enhancement in the various assays. For the stapled modifications, PA-0155 showed a significant increase in complement inhibitory activity. However, while a few stapled peptides did show enhanced activity in the various assays, the majority of these peptides showed similar activity to the parent molecule. For the D-amino acid variants, PA-0165 and -0166 had notably increased activity in the various assays.

The results shown here demonstrate that the functional activities of the PIC1 molecules can be improved significantly with peptide stapling as well as the introduction of non-canonical amino acids. Of interest and surprise was the finding that such modifications can lead to the improvement of one or multiple functional activities of the PIC1 peptide. The ability to isolate peptides with different functional activities can potentially be utilized to target specific inflammatory diseases where dysregulated complement, neutrophil (MPO and NETosis) or oxidant activity plays a predominant role in pathogenesis.

### Example 2. Pharmacokinetic Data for Select Peptides

In this experiment, four peptides from Example 1 that demonstrated superior activity in the in vitro assays for complement, NETosis and MPO inhibition were selected for further study. These peptides were PA-0117, -0127, -0130, and -0133. The four peptides were infused into rats, in order to determine their pharmacokinetic profiles by inhibition of hemolysis and binding assays.

### Methods

Male Wistar rats with indwelling jugular catheters were administered PA-0117, -0127, -0130, and -0133 with groups of three animals receiving 20, 200 and 400 mg/kg of the compound as a single, bolus, IV infusion. A control group of rats received an infusion of saline IV. At various time points after infusion (0.5, 2, 5, 20, 60, 120, 240, 480, 1440 minute) an aliquot of blood was drawn and plasma isolated and frozen at −70° C. pending analysis. Animals were sacrificed after the terminal blood draw and a gross necropsy performed.

### Results

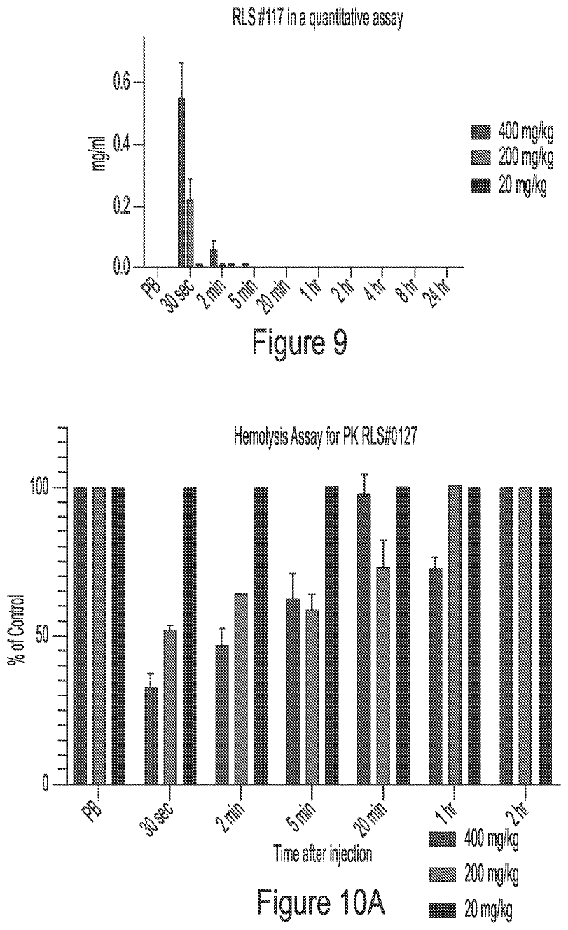

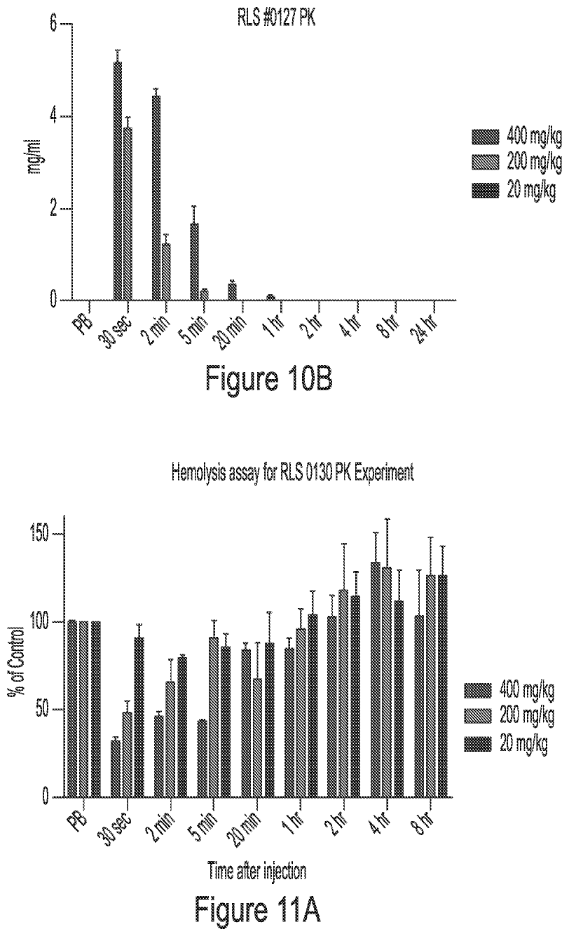

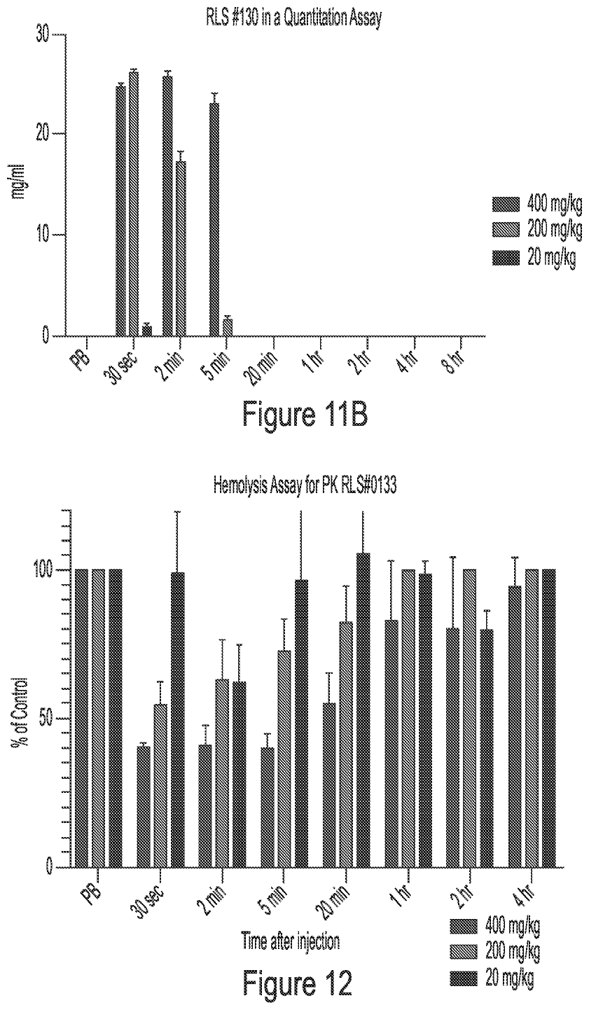

Rats receiving each peptide showed no signs of overt pathology by gross necropsy across all of the dose groups and there were no abnormalities noted in the blood chemistries or CBC analysis. Peptides PA-0127, -0130, -0133 all showed inhibition of complement activity in the hemolytic assay at the early time points at the highest dose and the inhibitory activity disappeared over time as expected (FIGS. 10A, 11A and 12). PA-0117 could not be analyzed as no functional complement activity was obtained from the plasma samples. For the quantitative (ELISA-like) target engagement assay in which the peptide levels in the samples are determined, different assays had to be used as the various peptides did not efficiently bind to C1q as the capture substrate and/or were not efficiently bound by the antibody used for detection. PA-0117 was analyzed in a C1q target acquisition assay (C1q binding assay) as described in Example 1 (FIG. 9). PA-0117 was detected at early time points at each dose and decreased over time as expected. PA-0127 and -0130 levels were determined by coating the plate with the plasma samples and directly detecting the peptides by rabbit antibody raised against PA-0020 (peptide sequence IAILILEPICCQERAA; SEQ ID NO: 57) (FIGS. 10B and 11B). As with PA-0117, increased binding was observed at early time points with diminished signal at later time points. PA-0133 could not be detected by the available antibodies.

### Data Summary

PA-0117: The hemolysis assay results were not useable (few pre-bleeds had any activity, and only a few other random samples had any complement activity). The PK assay for PA-0117 was performed by sandwich ELISA (FIG. 9).
PA-0127: The results of the hemolysis assay are discussed herein (see also FIG. 10A). For the PK assay, the sample was directly coated on a plate and then rabbit anti-PA was used, as the peptide did not bind well enough to C1q or chicken anti-I8 to use original assays (FIG. 10B).
PA-0130: The results of the hemolysis assay are discussed herein (see also FIG. 11A). For the PK assay, the sample was directly coated on a plate and then rabbit anti-PA was used, as the peptide did not bind well enough to C1q or chicken anti-I8 to use original assays (FIG. 11B).
PA-0133: The results of the hemolysis assay are discussed herein (FIG. 12). The PK assay was unable to be completed as the peptide did not interact with any of the anti-PA antibodies previously generated.

### Conclusion

The four selected peptides were active in vivo as assessed by inhibition of complement activity or C1q binding.

### Example 3. Inhibition of PD-1 Binding to PD-L1

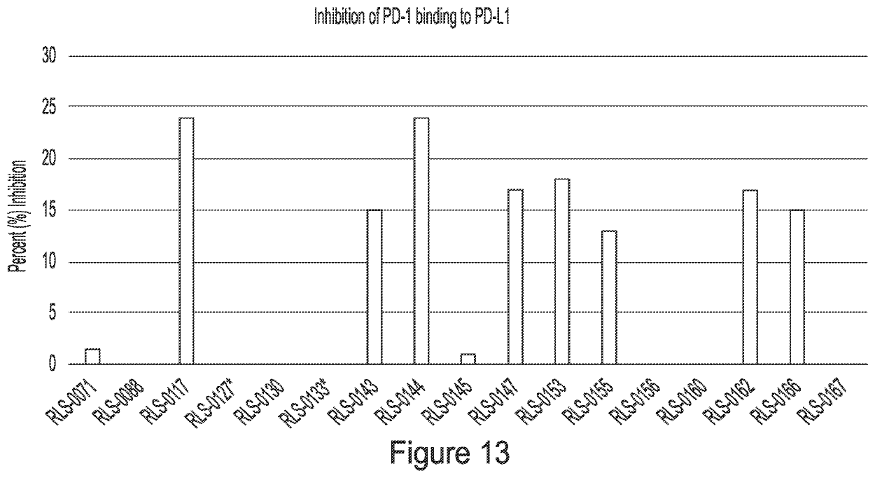

The immune check point pathway is an area of significant interest in cancer research. PD-1 is one of the best characterized checkpoint proteins. The binding between PD-1 and its ligand PD-L1 suppresses T-cell activation and allows cancer cells to escape from the body's immune surveillance. PD-L1 is a 40 kDa type 1 transmembrane protein that suppresses the adaptive arm of immune system during particular events such as pregnancy, tissue allografts, autoimmune disease and other disease states. A number of human cancer cells express high levels of PD-L1, and blockade of this receptor has been shown to reduce the growth of tumors in the presence of immune cells thus allowing tumor cells to evade anti-tumor immunity. Thus, PD-L1/PD-1 is a therapeutic target in cancer immunotherapy. The inventors evaluated whether the PIC1 peptides possessed the ability to inhibit binding of PD-1 to its receptor PD-L1 by using a commercial ELISA kit was utilized. PIC1 peptides demonstrated varying levels of inhibition of binding from 0%-24% (FIG. 13). This data is summarized in Table 4 demonstrating the ability of these peptides to inhibit PD-1 interaction with PD-L1.

### Example 4. Activity of PIC1 Peptides in a PD-1 Blockade Bioassay

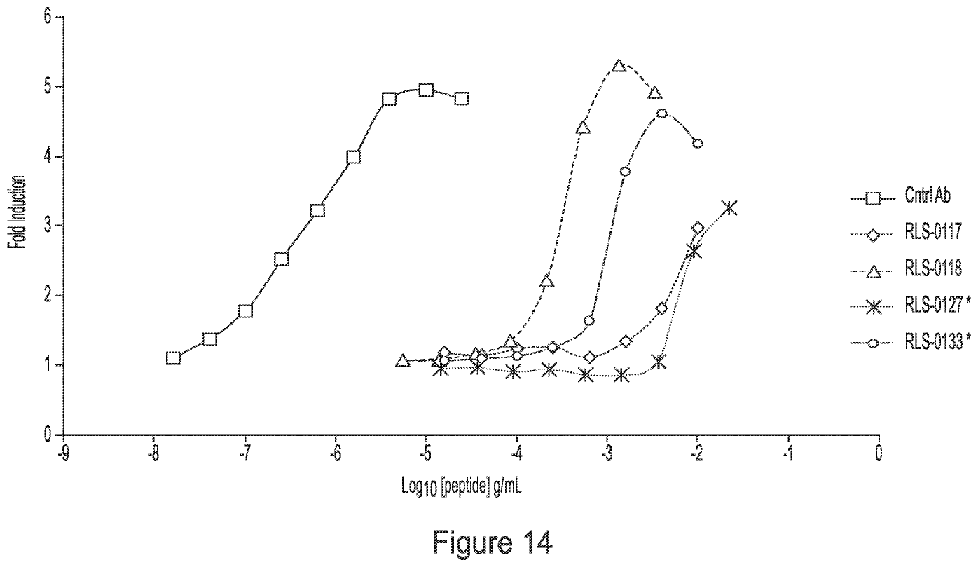

To assess if PIC1 peptides could block PD-1 inhibitory activity in a cell-based assay, select peptides were screened in the PD-1/PD-L1 Blockade Bioassay (Promega). The PD-1/PD-L1 Blockade Bioassay is a biologically relevant MOA-based assay that can be used to measure the potency and stability of antibodies and other biologics designed to block the PD-1/PD-L1 interaction. This bioluminescent cell-based assay is used to measure the potency and stability of molecules targeting PD-1 and consists of two genetically engineered cell lines: aAPC/CHO-K1 Cells are CHO-K1 cells with an engineered cell surface protein designed to activate cognate TCRs in an antigen-independent manner. When the two cell types are co-cultured, the PD-1/PD-L1 interaction inhibits TCR signaling and NFAT-mediated luciferase activity. Addition of an inhibitory molecule that blocks either PD-1 or PD-L1 releases the inhibitory signal and results in TCR signaling and NFAT-mediated luciferase activity. As with the anti-PD-1 antibody (positive control), select PIC1 peptides were screened in this assay with RLS-0117, RLS-0118, RLS-0127* and RLS-0133* demonstrating an increase in luminescence indicative of inhibition of PD-1/PD-L1 inhibitory signaling (FIG. 14). In contrast, RLS-0115, RLS-0122, RLS-0130, RLS-0142, RLS-0143, RLS-0144, RLS-0150, RLS-0154, RLS-0155, RLS-0156, RLS-0162, RLS-0164, RLS-0168, RLS-0170, RLS-0172, RLS-0173, RLS-0174 and RLS-0175 did not show PD-1/PD-L1 inhibitory activity in this assay (summarized in Table 4).

TABLE 4

| | | | | | | T-cell | T-Cell | T-Cell | | | |
|---|---|---|---|---|---|---|---|---|---|---|---|
| | SEQ. | PD-1 | PD-1 | CTLA-4 | CTLA-4 | Caspase | IL-2 | IFN-γ | VEGF | VEGF | Angiogenes |
| Name | ID NO. | Binding | Bioassay | Binding | Bioassay | inhibition | secretion | secretion | Binding | Bioassay | is inhibition |
| RLS- | 6 | ND | ND | ND | ND | ND | ND | ND | ND | ND | ND |
| 0114 | | | | | | | | | | | |
| RLS- | 7 | ND | − | ND | − | ++++ | − | − | ++++ | − | + |
| 0115 | | | | | | | | | | | |
| RLS- | 8 | ND | ND | ND | ND | ND | ND | ND | ND | ND | ND |
| 0116 | | | | | | | | | | | |
| RLS- | 9 | +++ | ND | ++ | − | ++++ | ++ | ++ | + | − | ++ |
| 0117 | | | | | | | | | | | |
| RLS- | 10 | ND | +++ | ND | − | +++ | + | + | + | − | +++ |
| 0118 | | | | | | | | | | | |
| RLS- | 11 | ND | ND | ND | ND | ND | ND | ND | ND | ND | ND |
| 0119 | | | | | | | | | | | |
| RLS- | 12 | ND | ND | ND | ND | ND | ND | ND | +++ | ND | ND |
| 0120 | | | | | | | | | | | |
| RLS- | 13 | ND | ND | ND | ND | ND | ND | ND | ND | ND | ND |
| 0121 | | | | | | | | | | | |
| RLS- | 14 | ND | ND | ND | ND | ND | ND | ND | ND | ND | ND |
| 0123 | | | | | | | | | | | |
| RLS- | 15 | ND | ND | ND | ND | ND | ND | ND | ++ | ND | ND |
| 0124 | | | | | | | | | | | |
| RLS- | 16 | ND | ND | ND | ND | ND | ND | ND | ++ | ND | ND |
| 0125 | | | | | | | | | | | |
| RLS- | 17 | ND | ND | ND | ND | ND | ND | ND | +++ | ND | ND |
| 0126 | | | | | | | | | | | |
| RLS- | 18 | − | ++ | ND | ND | ND | ND | ND | ND | ND | ND |
| 0127 | | | | | | | | | | | |
| RLS- | 54 | ND | ND | ND | + | +++ | ++ | +++ | ++ | − | + |
| 0127* | | | | | | | | | | | |
| RLS- | 19 | ND | ND | ND | ND | ND | ND | ND | +++ | ND | ND |
| 0128 | | | | | | | | | | | |
| RLS- | 20 | ND | ND | ND | ND | ND | ND | ND | +++ | ND | ND |
| 0129 | | | | | | | | | | | |
| RLS- | 21 | − | − | ND | ++ | ++++ | ++++ | +++ | ++ | − | ++ |
| 0130 | | | | | | | | | | | |
| RLS- | 22 | ND | ND | ND | ND | ND | ND | ND | +++ | ND | ND |
| 0131 | | | | | | | | | | | |
| RLS- | 23 | ND | ND | ND | ND | ND | ND | ND | +++ | ND | ND |
| 0132 | | | | | | | | | | | |
| RLS- | 24 | − | +++ | ND | ND | ND | ND | ND | ND | ND | ND |
| 0133 | | | | | | | | | | | |
| RLS- | 55 | ND | ND | ND | − | +++ | + | + | + | − | +++ |
| 0133* | | | | | | | | | | | |
| RLS- | 25 | ND | ND | ND | ND | ND | ND | ND | ND | ND | ND |
| 0135 | | | | | | | | | | | |
| RLS- | 26 | ND | ND | ND | ND | ND | ND | ND | + | ND | ND |
| 0136 | | | | | | | | | | | |
| RLS- | 27 | ND | ND | ND | ND | ND | ND | ND | ND | ND | ND |
| 0137 | | | | | | | | | | | |
| RLS- | 28 | ND | ND | ND | ND | ND | ND | ND | ND | ND | ND |
| 0138 | | | | | | | | | | | |
| RLS- | 29 | ND | ND | ND | ND | ND | ND | ND | ND | ND | ND |
| 0139 | | | | | | | | | | | |
| RLS- | 30 | ND | ND | ND | ND | ND | ND | ND | ND | ND | ND |
| 0140 | | | | | | | | | | | |
| RLS- | 31 | ND | ND | ND | ND | ND | ND | ND | ND | ND | ND |
| 0141 | | | | | | | | | | | |
| RLS- | 32 | + | − | ND | ND | ND | ND | ND | ND | ND | ND |
| 0143 | | | | | | | | | | | |
| RLS- | 33 | +++ | − | ND | ND | ND | ND | ND | ND | ND | ND |
| 0144 | | | | | | | | | | | |
| RLS- | 34 | − | ND | ND | ND | ND | ND | ND | + | ND | ND |
| 0145 | | | | | | | | | | | |
| RLS- | 35 | ND | ND | ND | ND | ND | ND | ND | ND | ND | ND |
| 0146 | | | | | | | | | | | |
| RLS- | 36 | ++ | ND | ND | ND | ND | ND | ND | ND | ND | ND |
| 0147 | | | | | | | | | | | |
| RLS- | 37 | ND | ND | ND | ND | ND | ND | ND | ND | ND | ND |
| 0148 | | | | | | | | | | | |
| RLS- | 38 | ND | ND | ND | ND | ND | ND | ND | + | ND | ND |
| 0149 | | | | | | | | | | | |
| RLS- | 39 | ND | ND | ND | ND | ND | ND | ND | ND | ND | ND |
| 0151 | | | | | | | | | | | |
| RLS- | 40 | ++ | ND | ND | ND | ND | ND | ND | ND | ND | ND |
| 0153 | | | | | | | | | | | |
| RLS- | 41 | + | − | ND | ND | ND | ND | ND | + | ND | ND |
| 0155 | | | | | | | | | | | |
| RLS- | 42 | − | − | ND | + | ND | ND | ND | ++++ | − | ND |
| 0156 | | | | | | | | | | | |
| RLS- | 43 | ND | ND | ND | ND | ND | ND | ND | ++++ | − | ND |
| 0157 | | | | | | | | | | | |
| RLS- | 44 | ND | ND | ND | ND | ND | ND | ND | +++ | ND | ND |
| 0158 | | | | | | | | | | | |
| RLS- | 45 | ND | ND | ND | ND | ND | ND | ND | ++++ | ND | ND |
| 0159 | | | | | | | | | | | |
| RLS- | 46 | − | ND | ND | ND | ND | ND | ND | ++++ | ND | ND |
| 0160 | | | | | | | | | | | |
| RLS- | 47 | ND | ND | ND | ND | ND | ND | ND | ++ | − | ND |
| 0161 | | | | | | | | | | | |
| RLS- | 48 | ++ | − | ND | ND | ++ | ++ | + | +++ | ND | ND |
| 0162 | | | | | | | | | | | |
| RLS- | 49 | ND | ND | ND | ND | ND | ND | ND | ++++ | − | ND |
| 0163 | | | | | | | | | | | |
| RLS- | 50 | ND | ND | ND | ND | ND | ND | ND | ++++ | − | ND |
| 0165 | | | | | | | | | | | |
| RLS- | 51 | + | ND | ND | ND | ND | ND | ND | ++++ | ND | ND |
| 0166 | | | | | | | | | | | |
| RLS- | 52 | − | ND | ND | ND | ND | ND | ND | ND | ND | ND |
| 0167 | | | | | | | | | | | |
| RLS- | 53 | − | ND | ND | ND | ND | ND | ND | + | ND | ND |
| 0169 | | | | | | | | | | | |

### Example 5. Binding of CTLA-4 by PIC1 Peptide RLS-0117

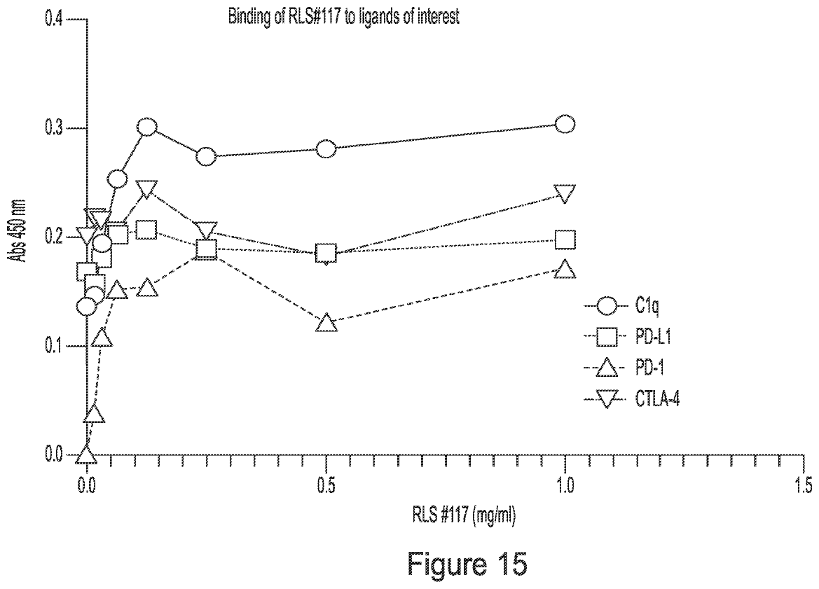

The inventors further evaluated if RLS-0117 could bind to the well characterized checkpoint inhibitor, cytotoxic T-lymphocyte associated protein 4 (CTLA-4 aka CD152). As with PD1/PD-L1 interactions, CTLA-4 is a check point protein that is often upregulated on the surface of cancer cells, engages ligands CD80 or CD86 on the surface of T-cells, and suppresses T-cell activation, allowing the cancer cells to escape destruction by the immune system. Therefore, the pharmaceutical inhibition of CTLA-4 or its ligand has been considered a promising strategy by many cancer researchers and is a therapeutic target in cancer immunotherapy. To ascertain the ability of RLS-0117 to bind CTLA-4, a binding assay was performed in which these proteins were coated on a microtiter plate followed by incubation with increasing amounts of either RLS-0134 and RLS-0150. Plates coated with PD-1, PD-L1 and C1q served as a positive control for peptide binding for peptide binding. RLS-0117 showed dose-dependent binding to PD-1, PD-L1 and C1q as expected and also bound CTLA-4 (FIG. 15) The ability of RLS-0117 to bind to CTLA-4 suggests that it may be able to functionally inhibit these interactions in cell-based bioassays.

### Example 6. Activity of PIC1 Peptides in a CTLA-4 Blockade Bioassay

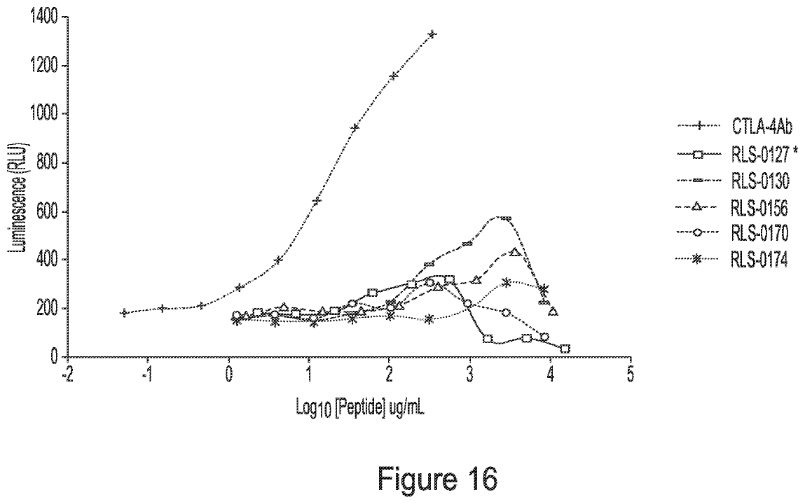

To assess if PIC1 peptides could block CTLA-4 inhibitory activity in a cell-based assay, select peptides were screened in the CTLA-4 Blockade Bioassay (Promega). This bioluminescent cell-based assay is used to measure the potency and stability of molecules targeting CTLA-4 and consists of two genetically engineered cell lines: CTLA-4 effector cells: Jurkat T cells expressing human CTLA-4 and a luciferase reporter driven by a native promoter which responds to TCR/CD28 activation and aAPC/Raji Cells: Raji cells expressing an engineered cell surface protein designed to activate cognate TCRs in an antigen-independent manner and endogenously expressing CTLA-4 ligands CD80 and CD86. When the two cell types are co-cultured, CTLA-4 competes with CD28 for their shared ligands, CD80 and CD86 and thus inhibits CD28 pathway activation and promoter-mediated luminescence. Addition of a molecule that blocks the interaction of CTLA-4 with its ligands CD80 and CD86 results in promoter-mediated luminescence. CTLA-4 antibody used as a positive control showed a dose-dependent increase in luminescence indicative of inhibition of CTLA-4 binding to its cognate receptor (FIG. 16). PIC1 peptides RLS-0127*, RLS-0130, RLS-0156, RLS-0170 and RLS-0174 all showed inhibitory activity in this blockade bioassay whereas RLS-0115, RLS-0117, RLS-0118, RLS-0133*, RLS-0156, RLS-0172, RLS-0173 and RLS-0175 peptides did not. These data are summarized in Table 4.

### Example 7. Inhibition of T Cell Exhaustion by PIC1 Peptides

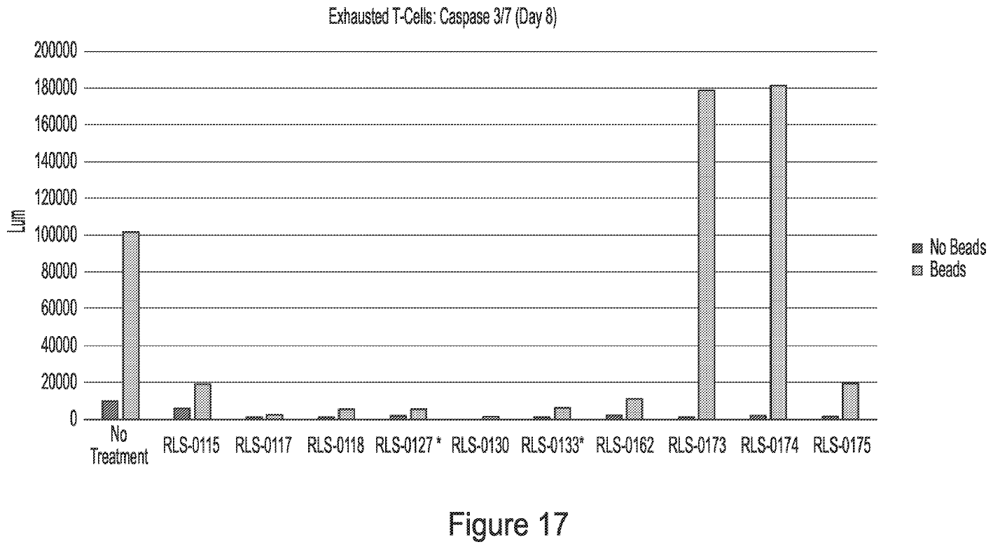

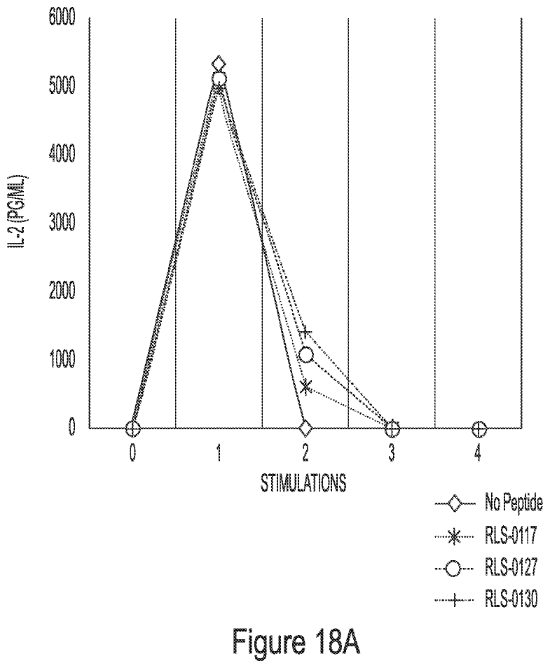

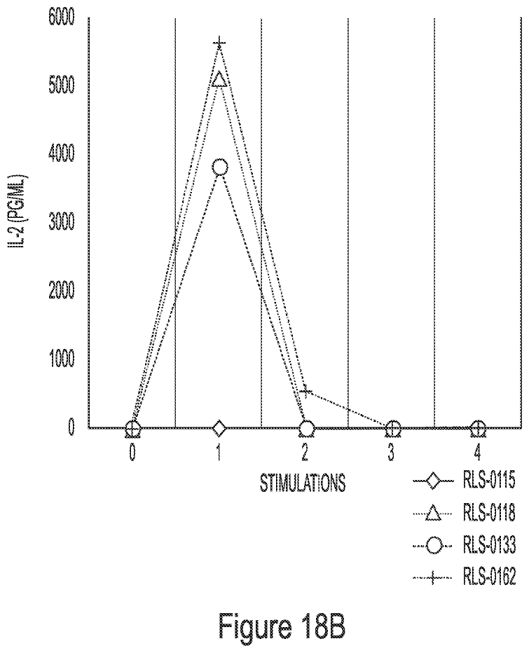

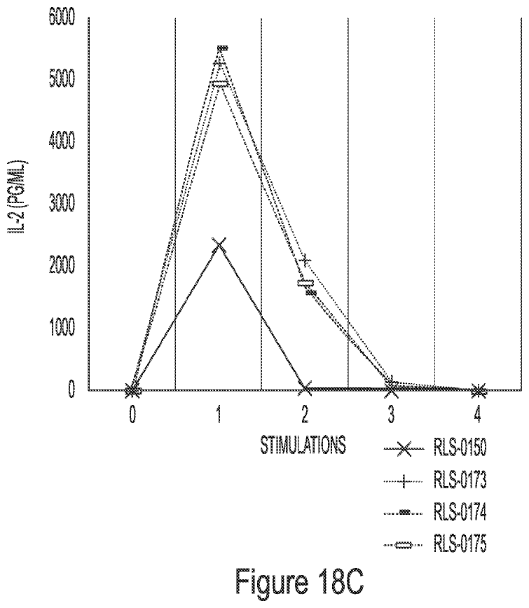

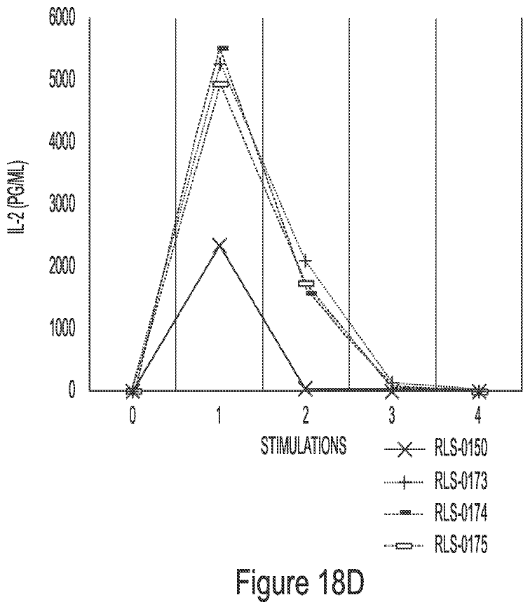

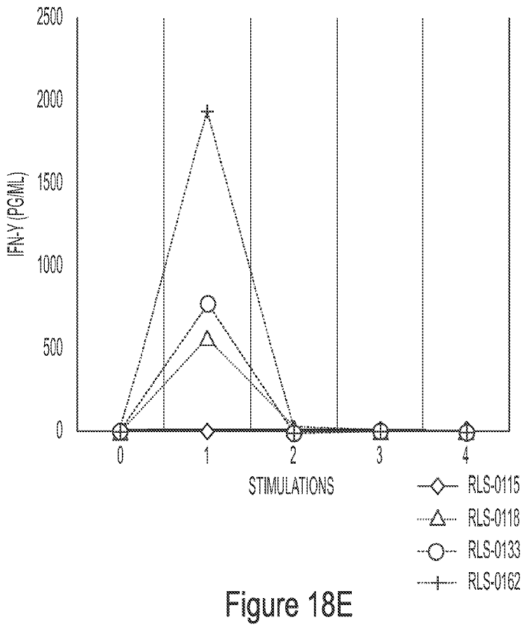

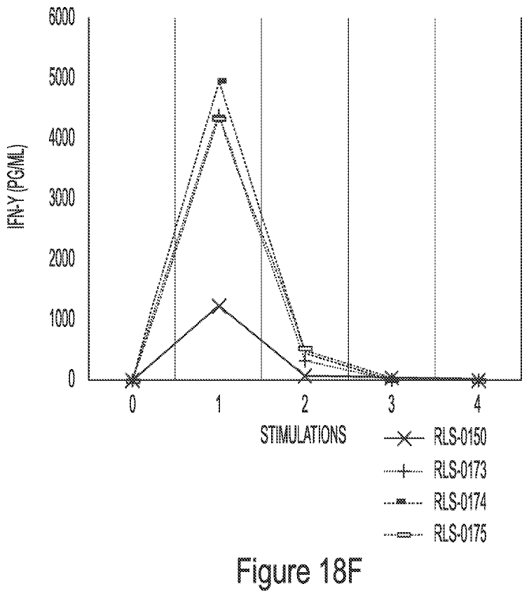

T-cell exhaustion is a form of T-cell dysfunction that occurs in cancer. It is defined by poor effector function, reduction in cytokine release (e.g., IL-2, TNF-alpha, IFN-gamma), sustained expression of inhibitory receptors (e.g., PD-1, LAG-3, CD244, CD160) with the progressive loss of effector function due to overstimulation. Exhaustion prevents optimal control of tumor growth. Exhausted T cells are prevalent in the tumor microenvironment (TME) and can lead to T-cell apoptosis. T-cell exhaustion is reversible, and pharmaceutical inhibition of T-cell exhaustion has been considered a promising strategy by many cancer researchers. To ascertain if PIC1 peptides could reverse T-cell exhaustion and increase T cell viability and effector function, a T-cell exhaustion protocol was developed. To induce T-cell exhaustion, purified human Pan T-cells were stimulated with T-Activator CD3/CD28 Dynabeads and cells are washed and re-stimulated every 48 hours. PIC1 peptides were added to the cells after each stimulation with Dynabeads. After three to four stimulations cells were harvested for readouts which consisted of assessing T-cell apoptosis by measuring Caspase 3/7 levels and production of cytokines IL-2 and IFN-gamma which is indicative of T-cell functionality. T-cells receiving bead stimulation in the absence of peptide showed an increase in Caspase 3/7 levels indicative of apoptosis (No treatment, FIG. 17). T-cells treated with PIC1 peptides showed decreased levels of Caspase 3/7 with some peptides such as RLS-0117 and RLS-0130 showing very low to undetectable levels of Caspase 3/7. In contrast, RLS-0173 and RLS-0174 showed increased levels of Caspase 3/7. To further evaluate if PIC1 peptides could restore T-cell functionality in cells that have undergone the exhaustion protocol, the inventors next assessed production of cytokines IL-2 and IFN-gamma. Supernatants from the cells after each stimulation were collected and the cytokines measured by ELISA. As shown in FIG. 18A-18C, cells receiving no peptide had a spike of IL-2 signal at Dynabead stimulation 1 which was then not detectable by stimulation 2. In contrast, cells treated with RLS-0117, RLS-0127*, RLS-0130, RLS-0162, RLS-0173, RLS-0174 and RLS-0175 showed an IL-2 signal at stimulation 2 whereas RLS-0115, RLS-0118 and RLS-0133* did not. When measuring IFN-gamma levels, the PIC1 peptides showed the same pattern of IFN-gamma release as seen with IL-2 (FIG. 18D-18F). The lower level of IFN-gamma signal compared to IL-2 was consistently seen in this assay. These data are summarized in Table 4.

### Example 8. Binding VEGF and Inhibition of VEGF Function by PIC1 Peptides

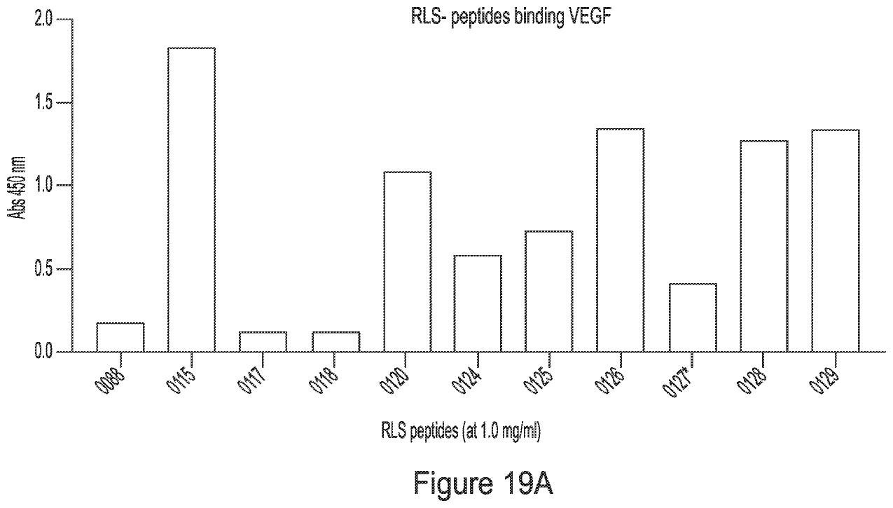

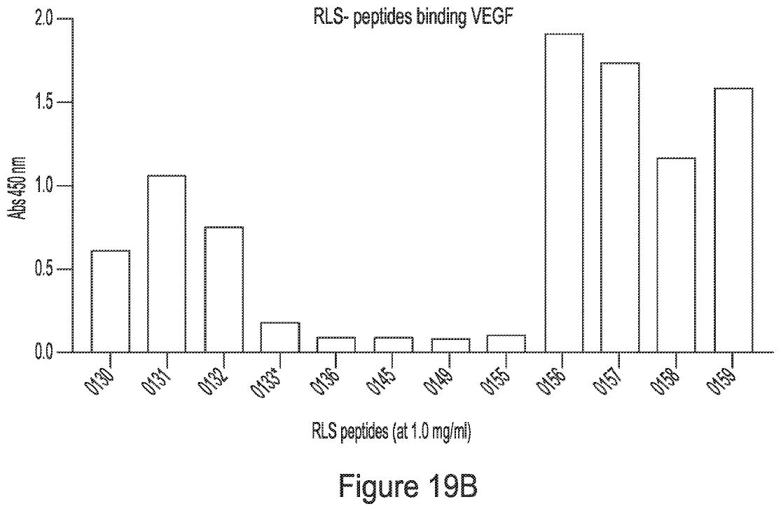

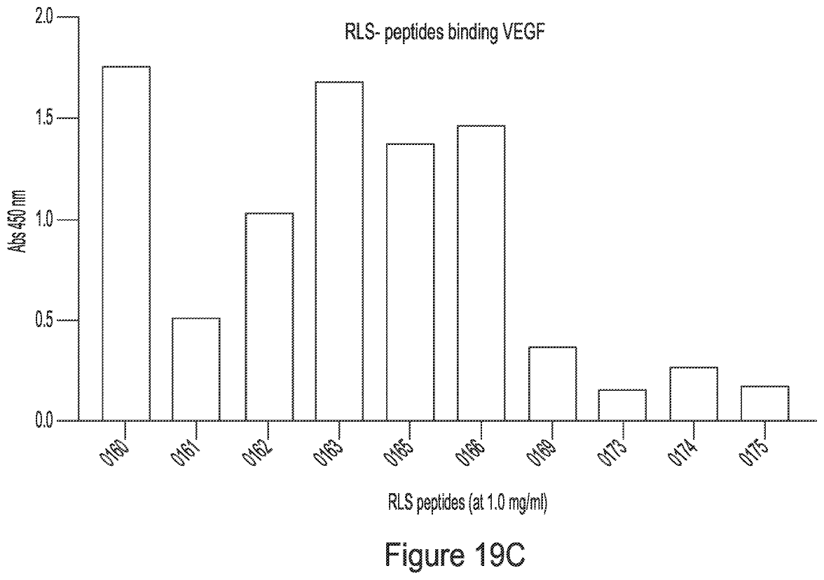

Angiogenesis, or the formation of new blood vessels from the established vasculature, is an essential element in tumor growth and metastasis formation. Inhibiting tumor angiogenesis is a major therapeutic strategy in oncology. Vascular endothelial growth factor (VEGF) is a potent and specific angiogenic factor and is a key requirement for tumor growth. VEGF inhibitors such as monoclonal antibodies are currently utilized to inhibit tumor growth in cancer patients. Although these anti-VEGF medications have proven to be effective for late-stage and metastatic cancers, they have been demonstrated to cause side effects such as hypertension, artery clots, complications in wound healing, and, more rarely, gastrointestinal perforation and fistulas. Thus, there is a need for safe VEGF inhibitors not based on monoclonal antibody technology. The inventors tested whether these PIC1 peptides possess the ability to bind human VEGF and inhibit VEGF function in a cell-based bioassay. To ascertain the ability of PIC1 peptides to bind VEGF, a binding assay was performed in which VEGF was coated on a microtiter plate followed by incubation with PIC1 peptides (1.0 mg/ml). As shown in FIG. 19A-19C, the PIC1 peptides bound VEGF at varying levels. Next, the inventors determined whether these PIC1 peptides could functionally inhibit VEGF mediated cell signaling via its cognate cell surface receptor, VEGFR-2 (KDR) using a VEGF bioassay (Promega). The VEGF Bioassay is a bioluminescent cell-based assay that measures VEGF stimulation and inhibition of VEGFR-2 using luciferase as a readout. This assay can be used for discovery and development of novel biologic therapies aimed at either inducing or inhibiting the VEGF response. The VEGF responsive cells have been engineered to express the response element (RE) upstream of luc2P, as well as exogenous VEGF receptor. When VEGF binds to VEGF responsive cells, the receptor transduces intracellular signals resulting in luminescence. The bioluminescent signal is detected in a luminometer. A select number of PIC1 peptides were tested in this bioassay (summarized in Table 4) but did not show inhibitory activity in this assay.

### Example 9. Inhibition of Non-VEGF Mediated Angiogenesis by PIC1 Peptides

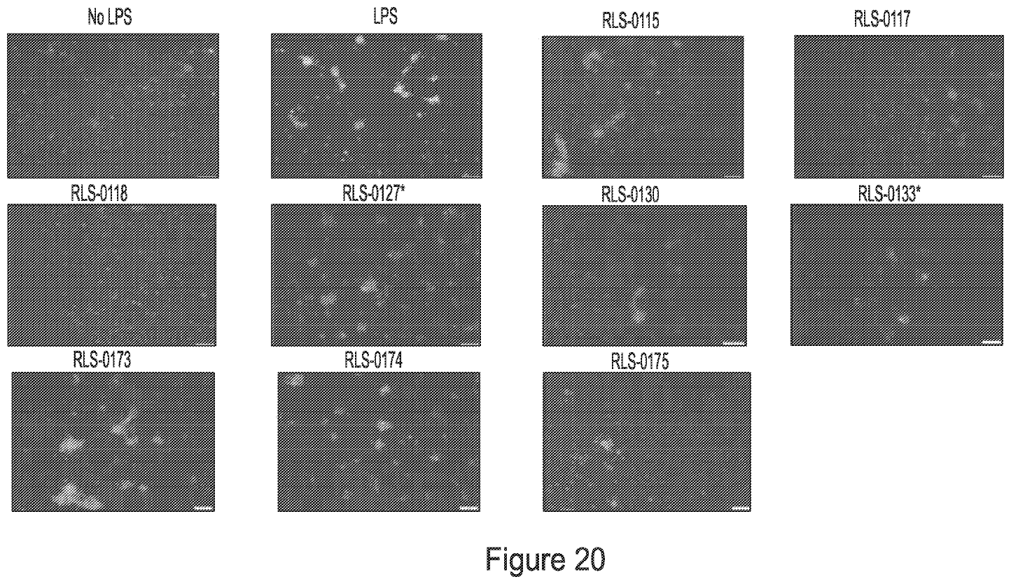

While VEGF-plays a major role in angiogenesis in cancer, other non-VEGF factors can induce angiogenesis to promote tumor growth. Currently there are no drugs on the market to inhibit non-VEGF mediated angiogenesis. To assess whether the PIC1 peptides could inhibit non-VEGF mediated angiogenesis, the inventors developed a model of angiogenesis using human umbilical endothelial vein cells (HUVECs) in which addition of lipopolysaccharide (LPS) induces angiogenesis. HUVECs were first incubated with Cell Trace Violet dye, followed by addition of PIC1 peptides (10 mg/ml) for 1 hour at 37° C. and then treated with 10 ug/ml of LPS and placement on an extracellular matrix to promote angiogenesis. Cells were incubated overnight at 37° C. in a humidified CO₂ incubator. Cells were then visualized for tube formation indicative of angiogenesis by fluorescence microscopy. Cells not receiving LPS did not show any clumping or formation of tube buds whereas these structures were evident in cells treated with LPS (FIG. 20). In the presence of PIC1 peptides, varying levels of angiogenesis inhibition were seen, with some peptides (RLS-0118 and RLS-0175) showing no signs of angiogenesis similar to control cells not stimulated with LPS. These data are summarized in Table 4.

### Example 10. RLS-0127* and RLS-0133* have Complement and Myeloperoxidase (MPO) Inhibitory Activity In Vitro

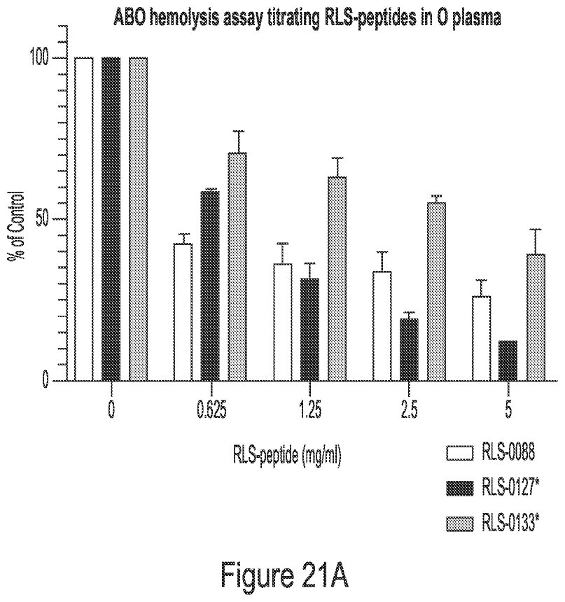

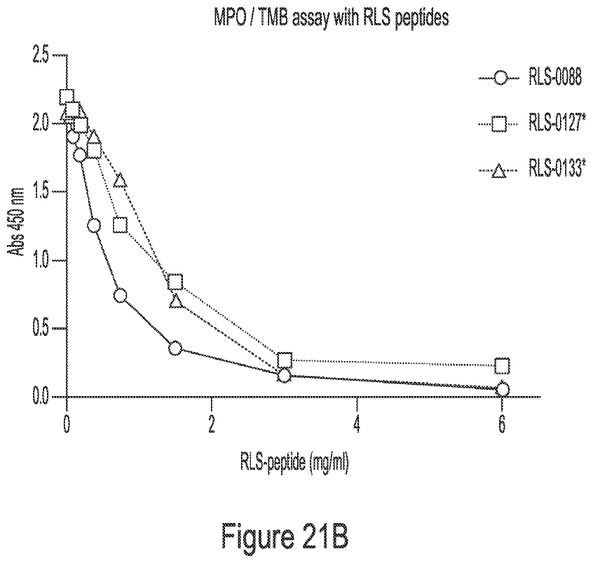

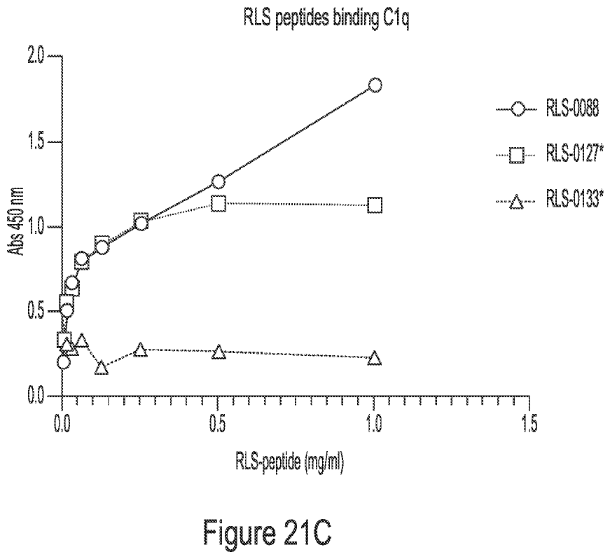

RLS-0127* and RLS-0133* when administered IV into Wistar rats were shown to inhibit complement activation in an ex vivo hemolytic assay (FIGS. 10A and 12) and pharmacokinetic data was also reported for RLS-0127* (FIG. 10B). The inventors further characterized the ability of these peptides to inhibit complement activation in vitro using human AB red blood cells sensitized with human O plasma. As shown in FIG. 21A, RLS-0127* and RLS-0133* dose-dependently inhibited complement activation, with RLS-0127* showing very similar levels of inhibition to the positive control, RLS-0088. The inventors also assessed the ability of RLS-0127* and RLS-0133* to inhibit MPO activity as determined by inhibition of oxidation of TMB in a plate-based assay. As with the hemolytic assay, RLS-0127* and RLS-0133* dose-dependently inhibited TMB oxidation in a similar manner to the positive control RLS-0088 (FIG. 21B). Additionally, the inventors assessed the ability of RLS-0127* and RLS-0133* to bind C1q in a plate-based assay in which C1q is coated on the plate and increasing amounts of the peptides are added, followed by primary antibody to the peptides and secondary antibody conjugated to HRP. The plate was then incubated with TMB to give a chemiluminescent signal that is detected at a wavelength of 450 nm in a plate reader. RLS-0127* bound C1q in a dose-dependent manner similarly to RLS-0088 (positive control) (FIG. 21C). RLS-0133 binding to C1q could not be detected as the primary antibody did not recognize this peptide. These data are summarized in Table 4.

### Example 11: Administration of Pharmaceutical Compositions

A pharmaceutical composition comprising a therapeutically effective amount of any of SEQ ID NOs: 6-55, and variants thereof, is administered to a subject in need thereof to regulate the complement system.

A pharmaceutical composition comprising a therapeutically effective amount of any of SEQ ID NOs: 6-55, and variants thereof, is administered to a subject in need thereof to inhibit myeloperoxidase activity.

A pharmaceutical composition comprising a therapeutically effective amount of any of SEQ ID NOs: 6-55, and variants thereof, is administered to a subject in need thereof to inhibit NETosis.

A pharmaceutical composition comprising a therapeutically effective amount of any of SEQ ID NOs: 6-55, and variants thereof, is administered to a subject in need thereof to inhibit oxidant activity.

A pharmaceutical composition comprising a therapeutically effective amount of any of SEQ ID NOs: 6-55, and variants thereof, is administered to a subject in need thereof to inhibit PD-1 binding to PD-L1.

A pharmaceutical composition comprising a therapeutically effective amount of any of SEQ ID NOs: 6-55, and variants thereof, is administered to a subject in need thereof to inhibit T cell exhaustion.

A pharmaceutical composition comprising a therapeutically effective amount of any of SEQ ID NOs: 6-55, and variants thereof, is administered to a subject in need thereof to inhibit angiogenesis.

### LIST OF EMBODIMENTS

The following is a non-exhaustive list of embodiments provided by the invention:
1. A synthetic peptide comprising at least about 95% sequence identity to an amino acid sequence selected from the group of SEQ ID NO: 6-55.
2. The synthetic peptide of embodiment 1 comprising an amino acid sequence selected from the group consisting of SEQ ID NO: 6-55.
3. The synthetic peptide of embodiment 1 comprising at least about 95% sequence identity to amino acid sequence selected from the group consisting of SEQ ID NOs: 9, 19, 22, 25.
4. The synthetic peptide of embodiment 1 comprising an amino acid sequence selected from the group consisting of SEQ ID NOs: 9, 19, 22, and 25.
5. The synthetic peptide of embodiment 1 consisting of an amino acid sequence comprising at least about 95% sequence identity to SEQ ID NO: 9.
6. The synthetic peptide of embodiment 1 consisting of an amino acid sequence comprising at least about 95% sequence identity to SEQ ID NO: 19.
7. The synthetic peptide of embodiment 1 consisting of an amino acid sequence comprising at least about 95% sequence identity to SEQ ID NO: 22.
8. The synthetic peptide of embodiment 1 consisting of an amino acid sequence comprising at least about 95% sequence identity to SEQ ID NO: 25.
9. A composition comprising at least one synthetic peptide of any of embodiments 1-8, optionally in further combination with another D-enantiomeric and/or stapled peptide form of SEQ ID NO: 4 and/or SEQ ID NO: 5, and variants thereof, and/or one or more of SEQ ID NO: 2, 3, 4, and/or 5, and variants thereof.
10. A pharmaceutical composition comprising a therapeutically effective amount of at least one synthetic peptide of any of embodiments 1-8 or the composition of embodiment 9, and optionally at least one pharmaceutically acceptable carrier, diluent, or excipient.
11. A method of regulating the complement system comprising administering the pharmaceutical composition of embodiment 10 to a subject in need thereof.
12. A method of inhibiting myeloperoxidase activity comprising administering the pharmaceutical composition of embodiment 10 to a subject in need thereof.
13. A method of inhibiting NETosis comprising administering the pharmaceutical composition of embodiment 10 to a subject in need thereof
14. A method of inhibiting oxidant activity comprising administering the pharmaceutical composition of embodiment 10 to a subject in need thereof.
15. A method of inhibiting PD-1 binding to PD-L1 comprising administering the pharmaceutical composition of embodiment 10 to a subject in need thereof.
16. A method of inhibiting T cell exhaustion comprising administering the pharmaceutical composition of embodiment 10 to a subject in need thereof.
17. A method of inhibiting angiogenesis comprising administering the pharmaceutical composition of embodiment 10 to a subject in need thereof.

While several possible embodiments are disclosed above, embodiments of the present invention are not so limited. These exemplary embodiments are not intended to be exhaustive or 10 to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

1. Fosgerau K and Hoffmann T (2014) Peptide therapeutics: current status and future directions. Drug Disc Today 20: 122-129.
2. Ali A M, Atmaj J, Van Oosterwijk N, Groves M R, Domling A (2019) Stapled peptide inhbitors: a new window for target drug discovery. Comp Struct Biotech J 17: 263-281
3. Sharp J A, Hair P S, Pallera H K, Kumar P S, Mauriello C T, et al. (2015) Peptide Inhibitor of Complement C1 (PIC1) Rapidly Inhibits Complement Activation after Intravascular Injection in Rats. PLoS ONE 10: e0132446.
4. Hair P S, Sass L A, Krishna N K, Cunnion K M (2017) Inhibition of Myeloperoxidase Activity in Cystic Fibrosis Sputum by Peptide Inhibitor of Complement C1 (PIC1). PLoS ONE 12: e0170203.
5. Hair P S, Cunnion K M, Krishna N K (2017) Peptide Inhibitor of Complement C1 Inhibits the Peroxidase Activity of Hemoglobin and Myoglobin. Int J Pept 2017: 9454583.
6. Gregory Rivera M, Hair P S, Cunnion K M, Krishna N K (2018) Peptide Inhibitor of Complement C1 (PIC1) demonstrates antioxidant activity via single electron transport (SET) and hydrogen atom transfer (HAT). PLoS ONE 13: e0193931.
7. Hair P S, Enos A I, Krishna N K, Cunnion K M (2018) Inhibition of Immune Complex Complement Activation and Neutrophil Extracellular Trap Formation by Peptide Inhibitor of Complement C1. Front Immunol 9: 558.
8. Hair P S, Rivera M G, Enos A I, Pearsall S E, Sharp J A, et al. (2017) Peptide Inhibitor of Complement C1 (PIC1) Inhibits Growth of Pathogenic Bacteria. International Journal of Peptide Research and Therapeutics DOI 101007/s10989-017-9651-z.
9. Matsui S M, Kiang D, Ginzton N, Chew T, Geigenmuller-Gnirke U (2001) Molecular biology of astroviruses: selected highlights. Novartis Found Symp 238: 219-233; discussion 233-216.
10. Bonaparte R S, Hair P S, Banthia D, Marshall D M, Cunnion K M, et al. (2008) Human astrovirus coat protein inhibits serum complement activation via C1, the first component of the classical pathway. J Virol 82: 817-827.
11. Hair P S, Enos A I, Krishna N K, Cunnion K M. (2019) Inhibition of complement activation, myeloperoxidase, NET formation and oxidant activity by PIC1 peptide variants. PLoS ONE 14: e0226875.
12. Cunnion K M, Lee J C, Frank M M (2001) Capsule production and growth phase influence binding of complement to Staphylococcus aureus. Infect Immun 69: 6796-6803.
13. Mauriello C T, Pallera H K, Sharp J A, Woltmann J L, Jr., Qian S, et al. (2013) A novel peptide inhibitor of classical and lectin complement activation including ABO incompatibility. Mol Immunol 53: 132-139.
14. Carlin J B, Doyle L W (2001) Statistics for clinicians: 4: Basic concepts of statistical reasoning: hypothesis tests and the t-test. J Paediatr Child Health 37: 72-77.
15. Rai J (2019) Peptide and Protein Mimetics by Retro and Retroinverso Analogs. Chem Biol Drug Des 93:7 24-736.

1. A synthetic peptide comprising at least about 95% sequence identity to an amino acid sequence selected from the group of SEQ ID NO: 6-55.

2. The synthetic peptide of claim 1 comprising an amino acid sequence selected from the group consisting of SEQ ID NO: 6-55.

3. The synthetic peptide of claim 1 comprising at least about 95% sequence identity to amino acid sequence selected from the group consisting of SEQ ID NOs: 9, 19, 22, 25.

4. The synthetic peptide of claim 1 comprising an amino acid sequence selected from the group consisting of SEQ ID NOs: 9, 19, 22, and 25.

5. The synthetic peptide of claim 1 consisting of an amino acid sequence comprising at least about 95% sequence identity to SEQ ID NO: 9.

6. The synthetic peptide of claim 1 consisting of an amino acid sequence comprising at least about 95% sequence identity to SEQ ID NO: 19.

7. The synthetic peptide of claim 1 consisting of an amino acid sequence comprising at least about 95% sequence identity to SEQ ID NO: 22.

8. The synthetic peptide of claim 1 consisting of an amino acid sequence comprising at least about 95% sequence identity to SEQ ID NO: 25.

9. A composition comprising at least one synthetic peptide of claim 1, optionally in further combination with another D-enantiomeric and/or stapled peptide form of SEQ ID NO: 4 and/or SEQ ID NO: 5, and variants thereof, and/or one or more of SEQ ID NO: 2, 3, 4, and/or 5, and variants thereof.

10. A pharmaceutical composition comprising a therapeutically effective amount of at least one synthetic peptide of claim 1, and optionally at least one pharmaceutically acceptable carrier, diluent, or excipient.

11. A method of regulating the complement system comprising administering the pharmaceutical composition of claim 10 to a subject in need thereof.

12. A method of inhibiting myeloperoxidase activity comprising administering the pharmaceutical composition of claim 10 to a subject in need thereof.

13. A method of inhibiting NETosis comprising administering the pharmaceutical composition of claim 10 to a subject in need thereof.

14. A method of inhibiting oxidant activity comprising administering the pharmaceutical composition of claim 10 to a subject in need thereof.

15. A method of inhibiting PD-1 binding to PD-L1 comprising administering the pharmaceutical composition of claim 10 to a subject in need thereof.

16. A method of inhibiting T cell exhaustion comprising administering the pharmaceutical composition of claim 10 to a subject in need thereof.

17. A method of inhibiting angiogenesis comprising administering the pharmaceutical composition of claim 10 to a subject in need thereof.

18. A pharmaceutical composition comprising a therapeutically effective amount of the composition of claim 9, and optionally at least one pharmaceutically acceptable carrier, diluent, or excipient.
